(19) United States
(12) Patent Application Publication
(10) Pub. No.: US 2026/0043017 A1
(43) Pub. Date: Feb. 12, 2026

(54) NANOCHANNEL LONG DNA ASSEMBLY AND SCALABLE END-TO-END AUTOMATION

(71) Applicant: Avery Bio Corporation, San Diego, CA (US)

(72) Inventors: Barry Merriman, La Jolla, CA (US); Ryan de Ridder, San Diego, CA (US); Laurence Warden, San Diego, CA (US); Someskumar Das, San Diego, CA (US); Matthew Holden, San Diego, CA (US); Himani Sharma, San Diego, CA (US); Janelle Lauffer, San Diego, CA (US); Sang Heon Lee, San Diego, CA (US)

(21) Appl. No.: 19/294,020

(22) Filed: Aug. 7, 2025

Related U.S. Application Data
(60) Provisional application No. 63/680,549, filed on Aug. 7, 2024.
(60) Provisional application No. 63/680,347, filed on Aug. 7, 2024.

(51) Int. Cl. C12N 15/10; B01L 3/00; C12Q 1/6813

(52) U.S. Cl. CPC C12N 15/10; B01L 3/5027; C12Q 1/6813; B01L 2200/14; B01L 2300/0861; B01L 2300/0896

(57) ABSTRACT

Provided herein are nanochannel devices fabricated in a substrate for holding an internal construct in a channel, wherein the construct comprises DNA, with the construct positioned so that a joinable end of the DNA is extending outside the channel into an accessible inlet reservoir. Also provided herein are methods for long DNA assembly, comprising introducing a shuttle with a block group in a nanochannel, introducing joinable DNA segment solution, performing a joining reaction, and advancing the shuttle in the channel while still leaving the tail in the channel.

### CROSS-REFERENCE TO RELATED APPLICATIONS

This Application claims priority to U.S. Provisional Patent Application No. 63/680,347 filed Aug. 7, 2024, entitled “NANOCHANNEL LONG DNA ASSEMBLY AND SCALABLE END-TO-END AUTOMATION,” and U.S. Provisional Patent Application No. 63/680,549 filed Aug. 7, 2024, entitled “NANOCHANNEL LONG DNA ASSEMBLY AND SCALABLE END-TO-END AUTOMATION,” each of which are entirely incorporated by reference for all purposes.

### FIELD

This disclosure relates generally to synthetic biology, and more particularly to nanochannel devices and methods for long DNA assembly. This disclosure relates to the fields of DNA synthesis, nanochannel technology, using nanochannel devices to assemble long DNA, semiconductor chip technology, and chip devices for carrying out long DNA assembly in nanochannels integrated into, and under the control of, a semiconductor chip device.

### BACKGROUND

Synthetic biology is often hailed as the foundation of the future US and global economy, as it will enabling engineer biology to transform the energy, manufacturing, mining, agriculture and health sectors, and to control the local and global environment. It is the central goal of synthetic biology to be able to program biological systems to carry out complex functions that achieve some desirable endpoint goal, such as designing microbes that produce valuable chemicals or clean the environment of pollutants, programming immune cells to kill cancer, or designing crops or livestock with desirable traits. All such control requires altering the gene content of cells, and for such complex functions, it can require changing many genes in an organism, or, ultimately, creating entire designer genomes. While modern genome editing methods such as CRISPR enable making many minor changes to existing genomes, the ultimate genetic control is to be able to directly fabricate very long DNA segments that contain many genes, or that may even constitute entire chromosomes or the entire genome of an organism. Such DNA segments may be in the size range of 10,000 bases up to millions or even hundreds of millions of bases in length. Current commercial DNA synthesis and assembly technologies can effectively produce single-gene length DNA, in the range of several thousand bases in length. Assembling these into longer segments is experimentally challenging and is only possible using complex, costly and time-consuming lab techniques. For example, the well-known method of Gibson Assembly (Gibson, Venter et al. “Enzymatic assembly of DNA molecules up to several hundred kilobases.” Nature Methods 6(5) (2009)) can assemble DNA of length up to hundreds of thousands of bases but is limited in length and content by the conditions of the complex solution-phase reaction, and the REXER method (Chin, Wang, et al. “Total synthesis of Escherichia coli with a recoded genome.” Nature 569 (7757) (2019)) can assemble million-base scale DNA, but only through a complex and lengthy serial process that must take place indirectly within a host cell. This process can take many months and cost millions of dollars to perform to make a full microbe genome.

Therefore, it is highly desirable for the future of synthetic biology to have effective means of assembling such very long DNA, that are free from length and content limits, are simple and fast to perform, and which can be fully automated and performed in scalable ways at low cost. The disclosures here provide such an efficient means of producing very long DNA for diverse uses in synthetic biology. Such a method of making long DNA is needed in order for synthetic biology to achieve its full potential as both a scientific field and as a transformative major industry.

### SUMMARY

In an aspect, a Nanochannel Long DNA Assembly Methodology is provided, as detailed herein, and is capable of producing very long DNA constructs (from 10 kilobase (kb) up to the 1 Megabase (Mb) range, or even more, up to 100 Mb range) of a precisely specified target sequence, built up from convenient short input “building-block” fragments (lengths typically in the 0.5 kb to 3 kb range, i.e. lengths that are convenient to produce with standard DNA synthesis and assembly techniques). In aspects, the method performs the DNA assembly steps by joining segments in nanochannels, using embedded electrodes to apply electrical forces to control the DNA motion in the channel. Assembling the long DNA in a nanochannel structure serves three critical, enabling purposes: (1) to prevent shearing motions that would otherwise break long DNA (2) to keep the end of the long DNA exposed and accessible for addition of the next segment, and (3) to enable a way for precision electrical motion control of the DNA needed to perform the joining operations. In order to achieve full automation on a scalable, highly parallel and low cost, manufacturable device, it is moreover most ideal to fabricate the nanochannels surface of standard semiconductor integrated circuit microchip as are made in current commercial chip foundries (specifically, CMOS chips), both as a means of (1) low cost mass manufacturing of such devices, by leveraging highly optimized chip foundry nanofabrication capabilities, and (2) as a means of having precision automation via the integrated electronic control circuitry available on such chips.

Also provided herein are nanochannel devices, comprising: a nanochannel fabricated in a substrate; configured to hold an internal DNA construct in the channel, with the construct positioned so that a joinable end of the DNA is in contact with an external reservoir that can be provided with additional DNA that can join to the joinable end.

Also provided herein are nanochannel devices, comprising: a nanochannel fabricated in a substrate, the nanochannel being configured to hold an internal DNA construct in the channel, with the construct positioned so that a joinable end of the DNA is extending outside the channel into an accessible inlet reservoir.

Also provided herein are methods for fabricating nanochannel assembly chips on a wafer, comprising: patterning nanochannels in a resist using electron beam lithography; etching the nanochannels using reactive ion etching; applying a photolithography resist; patterning microchannels into the photolithography resist; and forming by wet etching the nanochannels, the microchannels, and an inlet/outlet, wherein the inlet/outlet connects the microchannels and the nanochannels.

### BRIEF DESCRIPTION OF THE DRAWINGS

FIG. 1 shows the primary long assembly method.

FIG. 2 shows the long assembly method done in multiple channels.

FIG. 3 shows assembly done with multiple blocking groups.

FIG. 4 shows assembly done with multiple blocking groups.

FIG. 5 shows the time progression of assembly with multiple blocking groups.

FIG. 6 shows assembly done with multiple orthogonal blocking groups.

FIG. 7 shows assembly done with multiple orthogonal blocking groups.

FIG. 8 shows assembly done with multiple orthogonal blocking groups.

FIG. 9 shows the time progression of assembly with multiple orthogonal blocking groups.

FIG. 10 shows the synchronization of parallel assembly by use of multiple blocking groups.

FIG. 11 shows a lithography mask for a nanochannel assembly chip.

FIG. 12 shows a fabrication process for fabricating nanochannel assembly chips.

FIG. 13 shows a 3-D schematic CAD model view of a finished nanochannel assembly chip.

FIG. 14 shows a finished silicon wafer of passive nanochannel chip devices.

FIG. 15 shows a close-up view of finished nanochannel chip devices.

FIG. 16 shows the imaging station for experimental work on nanochannel chips.

FIG. 17 shows the field of view for single molecule imaging of the nanochannel chips.

FIG. 18 shows the imaging strategy for the nanochannel chips.

FIG. 19 shows a flow chart with steps of the automated imaging procedure for imaging the assembly process.

FIG. 20 shows an example mode of image acquisition and interpretation for imaging of the nanochannel chips.

FIG. 21 shows one preferred embodiment of the shuttle.

FIG. 22 shows steps of making the shuttle.

FIG. 23 shows steps of making the shuttle.

FIG. 24 shows steps of making the shuttle.

FIG. 25 shows a gel analysis of the shuttle products,

FIG. 26 shows shuttle molecules on display and loaded in channels.

FIG. 27 shows shuttle molecules on display in two colors.

FIG. 28 shows shuttle molecules on display in two colors.

FIG. 29 shows shuttle molecules loaded in channels.

FIG. 30 shows a preferred shuttle architecture.

FIG. 31 shows the component details for the shuttle architecture.

FIG. 32 shows the major shuttle assembly steps.

FIG. 33 shows a gel analysis showing the correct shuttle construction.

FIG. 34 shows a device schematic for transporting and storing long DNA.

FIG. 35 shows the experimental demonstration of long DNA transport and collection.

FIG. 36 shows a schematic of the passive chip for an automated rig.

FIG. 37 shows the assembly of the chip into a holder.

FIG. 38 shows and exploded view of the chip holder assembly.

FIG. 39 shows the fluid sample delivery stage.

FIG. 40 shows the autosampler schematic for the rig.

FIG. 41 shows how nanochannel devices can be supported by CMOS chip technology.

FIG. 42 shows a prefere3d embodiment for a chip architecture for the CMOS nanochannel chip.

### DETAILED DESCRIPTION

The starting point is a long DNA sequence design that is to be produced. From this design, the long sequence is broken up into short segments of a convenient length for primary production, such as in the range of 0.5 kb-3 kb. All of these (typically tens to hundreds or thousands) segments are first prefabricated (from any source of gene-length DNA, but ultimately on a separate oligo synthesis and gene assembly chip, for full end-to-end design-to-long DNA production). In particular, these ˜1 kb segments are primarily double-stranded DNA (dsDNA) but made with single stranded “sticky end” overhangs so that they would fit together by complementary sequence hybridization to form the desired long strand. The overhang length is chosen to provide highly stable hybridization in the process below, such as 30 base long overhangs, or preferably overhand in the 15-50 base range. These “building blocks” are suitably organized to be individually available as input reagents for the long assembly process (e.g., residing individually in the wells one or a few 96- or 384-well plates).

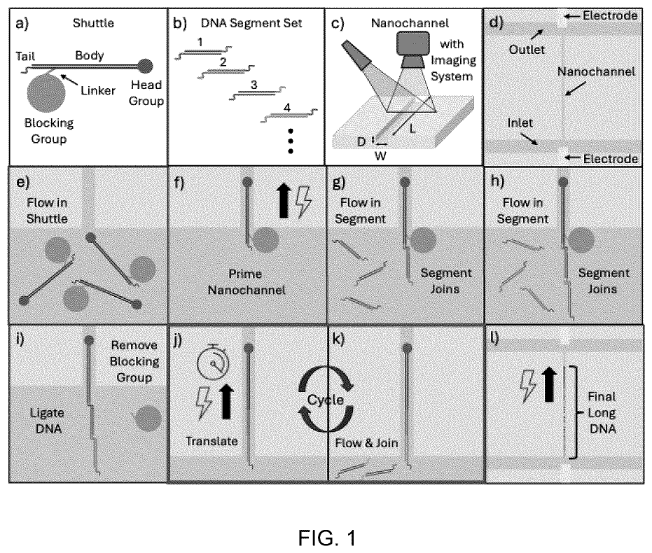

FIG. 1 illustrates the basic nanochannel long DNA assembly process to be disclosed here. This basic channel geometry is shown (FIG. 1c). The length of the nanochannel is mainly related to the length of DNA to be assembled. For example, a 1 Mb long DNA segment has an extended length of ˜0.3 mm, and so the long dimension of the channel would be (at least) ˜0.3 millimeters to be able to hold an entire completed construct. Making even longer fragments up to 10 Mb would require longer channels, at least ˜3 mm long. Making 100 Mb fragments would require channels at least ˜30 mm long. For such long channels, it may be advantageous to give them a serpentine or zig-zag geometry, so that they can fit on a chip whose lateral dimensions are less than 30 mm. The lateral confining dimensions of the channel are approximately 100 nanometers (nm) in width and depth to force the DNA to linearize and to constrain the motion to be along the channel. The width and depth dimensions in preferred embodiments range from as little as 10 nm to as much as several hundred nanometers. Within the overall workflow described below, the nanochannel serves to protect, constrain, linearize, orient, and position the DNA fragments, and allow the ends of segments to join using standard hybridization-based end-joining techniques (e.g. using “sticky end overhangs” for sequence-specific joining, followed by subsequent enzymatic ligation). FIG. 1c illustrates an embodiment where there may be an imaging system present to be able to record the assembly process using still images or video, for quality control, process control and analytical purposes. FIG. 1d illustrates a further embodiment shown in an overhead view, wherein there are inlet and outlet microchannels (with width and depth in the micron range to facilitate fluid flow and material transport to and from the nanochannel) as well as primary control electrodes in these reservoirs, which are used to apply an electric field that draws DNA into and through the nanochannel, based on the applying a voltage gradient that moves the negatively charged DNA. As detailed herein, it is possible to use the nanochannel to precisely organize serial segment additions that can be used to build up the desired long DNA with precision from the given short pieces.

Primary Process: The primary long DNA nanochannel assembly process is illustrated in FIG. 1 (figure panels FIG. 1e through FIG. 1l). The assembly process is initiated in a nanochannel, fabricated on the surface of a chip (or suitable hard and flat substrate, such as glass or silicon or plastic), between two microchannels, which act as input and output reservoirs, respectively (FIGS. 1c, 1d). In certain embodiments, the entire process is monitored by an imaging system (located above the device, FIG. 1c), that can be used for quality control purposes to observe proper formation of the long DNA construct in the channel, which can be made visible with dye stains.

After initial nanochannel preparation (e.g., wetting with a suitable buffer solution), the nanochannel is primed for DNA assembly by first loading in a universal “Shuttle” DNA segment (FIG. 1a), which are input via the microchannel and drawn into the nanochannel via a driving electric field acting on the negative charge of the Shuttle DNA body. The Shuttle is a custom DNA construct fabricated and optimized for this purpose. Generally, this is referred to as a “Shuttle” as it plays a role similar to that of the shuttle in a weaving, which guides the long thread through the weave. It is understood that the Shuttle may be constructed of any suitable polymer, including, but not limited to, DNA. For example, the Shuttle may be constructed of any other suitable nucleic acid. The Shuttle DNA is constructed with features (FIG. 1a) and modifications that enable efficient, oriented and synchronized loading of channels. As shown (FIG. 1a), in certain embodiments the Shuttle architecture is based on a standardized, conveniently long DNA “Body” molecule (e.g., a modified Lambda DNA, 50 kb long), that acts as a standard universal channel primer for all subsequent nanochannel assemblies, which will proceed by adding the desired DNA segments sequentially to the sticky ended tail of the Shuttle DNA. The other essential element is the Blocking Group near the tail, that is too large to fit in the nanochannel (e.g., a polymer bead in the range of ˜100 nm to ˜500 nm in diameter), so that the Shuttle can only be loaded into the nanochannel in one orientation, in which the head goes in first and the tail remains positioned extending into the microchannel, exposing a first sticky end at which to initiate DNA segment addition, and so that the Shuttle can be stopped and staged precisely at the channel entrance due to the blocking group forcing it to stop there at a precise location. The Blocking Group is coupled to the Body by a reversable linker so that it may be conveniently released. In certain embodiments, such a reversible or cleavable linker could be comprised of many well-known forms of linking molecules and reversable or cleavable groups. For example, the linker could be a carbon chain, a PEG chain, a single or double stranded DNA, or a peptide chain or protein, and the cleavable/reversable group could be a disulfide bond, a photo-cleavable group, a thermally cleavable group, a chemically cleavable group, an enzymatically cleavable group, a DNA that can be cleaved in a sequence-specific way by restriction enzymes or CRISPR Cas enzymes, or a hybridized DNA oligomer that can be de-hybridized through thermal melting, chemically induced melting, displacement by a displacing oligo of higher binding affinity, or removal by a strand displacing polymerase enzyme. The head of the Shuttle DNA Body may in preferred embodiments optionally be modified in useful ways, such as having a Head Group to carry added charge to enhance the electrical force that pulls the Shuttle within the channel, or a group that has friction in the channel to arrest unwanted DNA motion in the channel, or to carry a group that is brightly imaged, for tracking in imaging (such as dye molecules, a dye-labeled bead, a quantum dot, or a gold nanoparticle), and the Shuttle DNA Body may also be dye-labeled for imaging observation as well. It is assumed there is a set of segments to be joined, indicated in FIG. 1b, which can be made available in the system, serially as needed. For example, in preferred embodiments this segment library may reside in well plates or may be supplied by a microfluidic dispensing device coupled to the system.

To start the assembly process, a solution of Shuttle molecules is introduced to the microchannel (FIGS. 1d, 1e), and the (positive) voltage along the nanochannel is turned on to pull the (negative) charged DNA into the nanochannel. The Shuttle can only enter in the one desired “head-first” orientation, and only a single Shuttle can enter, due to the Blocking group. Once the nanochannel is primed (FIG. 1f) with a Shuttle, the voltage is kept on so that the Shuttle is held in place (or it may be held in some preferred embodiments by its friction or steric hinderance within the channel), and the remaining unloaded Shuttles are washed out of the microchannel. Next, the first double stranded DNA (dsDNA) segment, having a designed sticky end that specifically joins to the Shuttle tail sticky end, is introduced to the nanochannel through the microchannel (FIG. 1g). As the tail of the Shuttle is positioned extending into the microchannel, this hybridization is done in a standard bulk environment, and therefore with the standard high efficiency and rapidity of simple hybridization reactions. In particular, the speed and efficiency of this can readily be controlled by the concentration of the input DNA. After the hybridization is complete (typically in seconds, or at most a few minutes) the first dsDNA segment is washed out of the microchannel. This step of introducing subsequent dsDNA segments for sticky end joining can be repeated several times, causing the DNA construct to grow out further into the microchannel. After a series of one to several such hybridization joining steps (limited by not wanting this tail in the channel to get long enough to shear or ball up and obstruct access to the trailing sticky end), the Blocking Group is released (e.g., by flowing in a cleavage or reversing reagent), and an enzymatic ligation step may optionally be performed at this point to covalently link the segments (but this is not essential, as long as the designed-in sticky end hybridizations are chosen to be very stable, such as having a high melting point well above the ambient temperature, such as above 50° C. for a room temperature process and preferably above 80° C.).

As the Shuttle no longer has a Blocking Group impeding motion into the nanochannel, an electrical force (either electrophoretic (EP) or dielectrophoretic (DEP)) can be applied to the channel, pulling the Shuttle to move further into the nanochannel and thereby pulling the growing long DNA into the channel. The distance of translocation into the nanochannel can be precisely controlled by the time duration and magnitude of the applied driving voltage, and this can also be observed by the imaging system that monitors the Shuttle location in the channel.

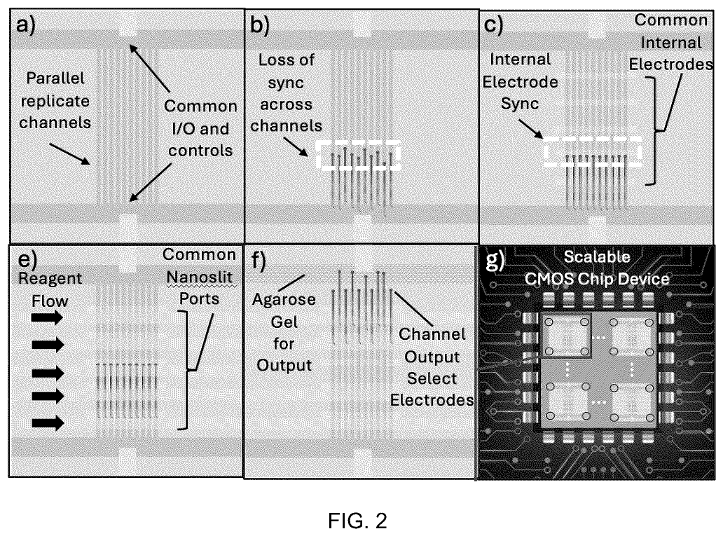

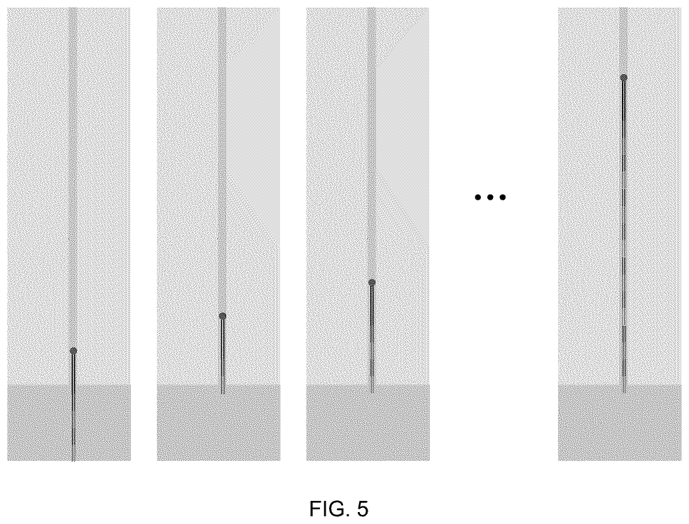

In certain other embodiments, as illustrated in FIGS. 2-10, internal electrodes or other blocking groups on the incoming segments can also be used to precisely control this translocation of the growing DNA into the channel. In any case, this translocation distance is to be controlled so that the one free sticky end of the trailing dsDNA segment remains extended into the microchannel as the site for subsequent joins. These steps of joining a next segment, ligation, and translocation into the nanochannel are then repeated serially (FIGS. 1j-1k) until the assembly of all desired input pieces is complete (typically tens to hundreds of cycles, and up to thousands or even tens of thousands of cycles as needed to achieve the desired final long DNA product). The result (FIG. 1l) is the final desired long DNA residing in the nanochannel and attached to the Shuttle, which is at the most advanced end of the entire construct in the nanochannel. In certain embodiments, the time required for this assembly is on the order of 1 minute per cycle, and thus the assembly of even Mb-scale DNA can be completed in 5-10 hours, depending on the number of pieces used. Shorter targets in the 100 kb range would take only 1-2 hours. The actual time required for each join may be in the range of seconds to minutes and can be controlled by the concentration of the joining segments.

Scalable Parallel Assembly Devices: The basic assembly process creates a single long DNA molecule in a single nanochannel. To obtain highly parallel and scalable device architectures, this unit process can readily be scaled out in parallel fashion, and integration onto a chip for scalable control electronics, as shown in FIG. 2. The first step towards the scaled architecture (FIG. 2a) is to carry out the basic process in parallel across a local group of “replication” nanochannels, wherein each nanochannel in the group is exposed to the same electrical control voltages and the same input solutions and DNA input segments, and therefore ideally assembles a replicate DNA molecule. The purpose for this replication is: (a) that it provides a convenient means to obtain multiple copies of the target long DNA molecule, and (b) provides a means to ensure sufficient yield of the target long DNA, given that some channels may fail to properly form the construct, due to various possible failure modes, such as failure to join a segment or a channel becoming clogged or fouled.

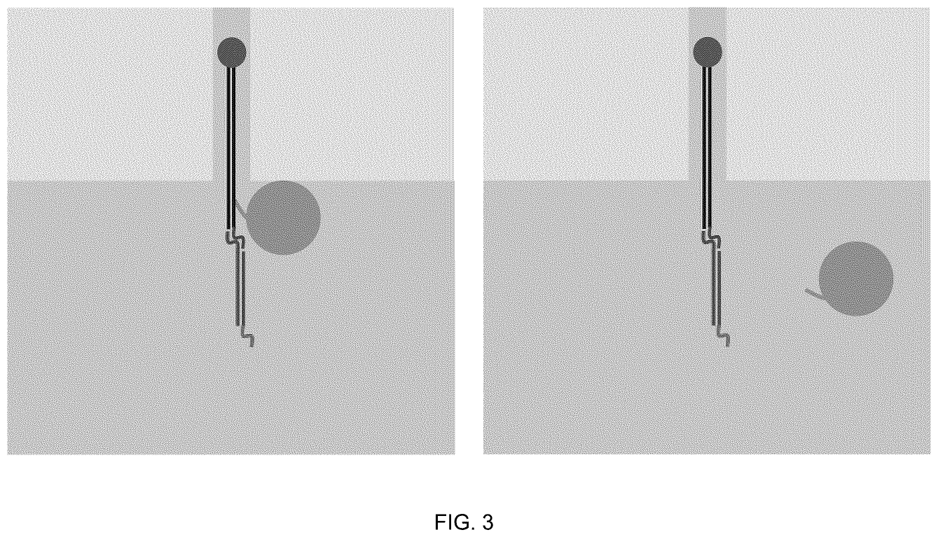

In the case of having replicate nanochannels, even though all of the Shuttles are positioned at the inlet of their channels identically at the start of assembly (due to the aligning effect of the block group positioned against the nanochannel inlet) as shown in FIG. 2b, the Shuttles will not remain this positionally aligned during assembly, due to minor device variations (e.g., nanochannel fabrication variations, electric field variations, fluid pressure variations, DNA diffusion and DNA Brownian motion etc.), and some will be traversing deeper into the nanochannels than others. To maintain the high precision control of assembly in this setting, it is desirable to have a means to re-synchronize the Shuttle locations. As shown in FIG. 2c, in one preferred embodiment this can be achieved by integrating common motion control electrodes, cutting across all channels in the group, at suitably spaced locations along the channels. One of these internal “strip” electrodes can exert a force that pull all Shuttles precisely to that location, thus re-synchronizing the Shuttle position in the channels. These provide a finer degree of position control than relying only on the timing of the common driving voltage. As shown in FIGS. 3-10, in other preferred embodiments additional block groups on the incoming segments can be used to synchronize shuttle locations across replicate channels by imposing uniform physical positioning stops in the assembly process.

Further sophisticated nanochannel features can be added to enable more robust operations and additional processing capabilities. Foremost of these is to add transverse “nanoslit” channels spaced along the length of the channels, that can be used to flow reagents in and out of the internal portions of the channels (FIG. 2e), without disrupting the long DNA in the channel. This is accomplished by making these cross channels very shallow, and cutting across the top of the nanochannels, for example just about 10 to about 40 nm deep atop an about 100 nm deep channel. As long as the Shuttle guide can traverse these nanoslits without leaving the nanochannel, the trailing long DNA will stay properly situated within the nanochannel. A new capability added by these fluidic access points is that enzymes can be introduced to operate on the DNA in the channel. For example, this can be used to repair damage by adding ligase to repair any nicks, or other DNA damage repair enzymes, polymerase can be added to reel of local DNA copy strands that can be collected for sequencing to verify accuracy of the long DNA sequence, or CRISPR or other editing systems can be introduced to perform error correction in place on the long DNA.

The most generic output process for the finished long DNA is to use the driving voltage to pull the DNA into an agarose gel in the output reservoir (FIG. 2f), as this “gel plug” protects the long DNA from shearing and is a compatible way to transfer it into other synthetic biology workflows, such as transfection into cells. Working with long DNA suspended in gel plugs is now a well-established storage and transfer format for such long DNA. The agarose is readily digested away for subsequent uses of the long DNA by the agarase enzyme. Because a channel may contain a failed assembly-as would be observed by the imaging system-it is desirable to add a means to only export into the transfer gel plug the DNA contents from select nanochannels with well-formed long DNA products. To provide this capability, these multichannel devices have each channel configured with an individual output control electrodes at the output end of the channel, such that-based on assembly quality control monitoring via the imaging system-only channels with well-formed long DNA will have their contents transferred to the output collection reservoir.

For ultimate device scalability, the above long assembly structures will be formed on the surface of a CMOS chip device (FIG. 2g), that provides for: (a) full integration and miniaturization of the all the necessary electronic circuitry that controls the assembly, and (b) the most ideal means of device fabrication, by enabling the use of standard CMOS foundries to make the entire chip device, both the CMOS circuity and the nanochannel structures. In extending these replicate nanochannel groups onto a CMOS chip die, which have standard sizes up to ˜30 mm×30 mm, there is ample room to have multiple, independently fluidically addressing channel bundles, such that one chip could readily support 10's-100's of independent channel replicate groups, each having its own independent input/output microchannels, and each of which of which itself internally comprises 10's to 100's of replicate channels. Thus, the CMOS devices can ultimately be capable of parallel assembly of 100's of entirely independent long DNA constructs, that can be made from whatever input fragments are provided, and where each such target long DNA construct is itself made in replicate in the 10's to 100's using the fine scale replication nanochannels. Notable for the ultimate future end-to-end process, going from long DNA design-to-production with full on-chip automation of all processes, this ideally matches the ultimate capacity of the long DNA oligo synthesis methods and gene-length DNA assembly chips disclosed herein, which could produce (and electrokinetically on-chip transport to the microchannel inputs) the up-to million-scale input segments needed to make hundreds to thousands of different, independently specified about 100 kb to about 1 Mb sequence constructs in parallel.

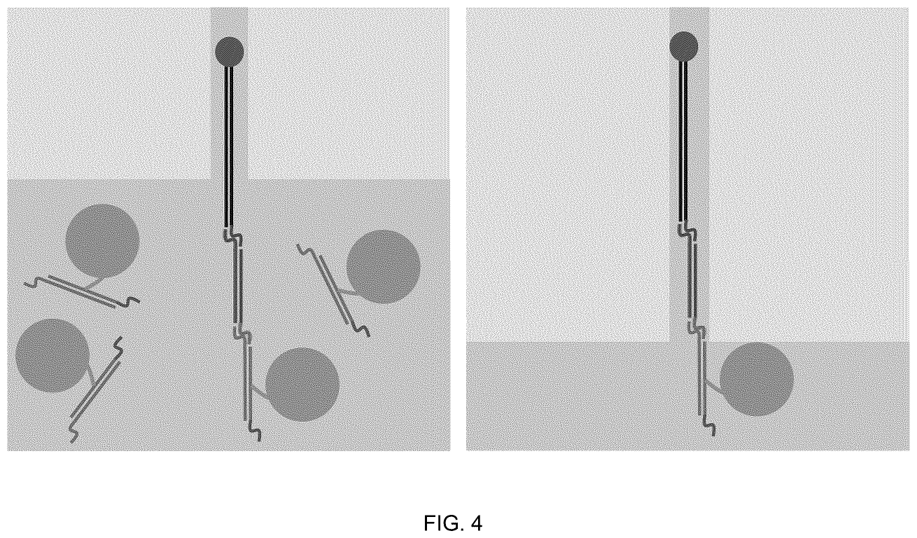

FIGS. 3 and 4 illustrate another embodiment of the long assembly process where additional blocking groups are used to precisely control the DNA translocation in the channel. This process is shown starting in FIG. 3, where (left) the Shuttle has been staged in the channel via its blocking group, and one segment has been joined to it. On the right is shown the blocking group being cleaved off, leaving the DNA strand in the channel. There is assumed to be sufficient friction between the channel and the shuttle that the DNA does not move significantly in the channel. Then, the next DNA segment to be joined is brought in via flowing in through the microchannel, and this segment also has the blocking group reversibly attached to it, such as it was for the Shuttle. As shown, one of these will join to the end of the segment in the channel, the rest can then be flushed out of the microchannel, and now the DNA can be translocated under the electrical force with no active control, because the blocking group will stop the motion at precisely the right point, leaving a joinable end exposed. Iterating this process, as shown in the time series of images in FIG. 4, will result in the growing strand advancing into the channel with precision position control, as defined by the physical stops provided by the blocking groups on the control segments. These control segments could be every segment, or they could be every other segment, or every third segment, or any reasonably frequent series of segments, frequent enough to retain adequate motion control.

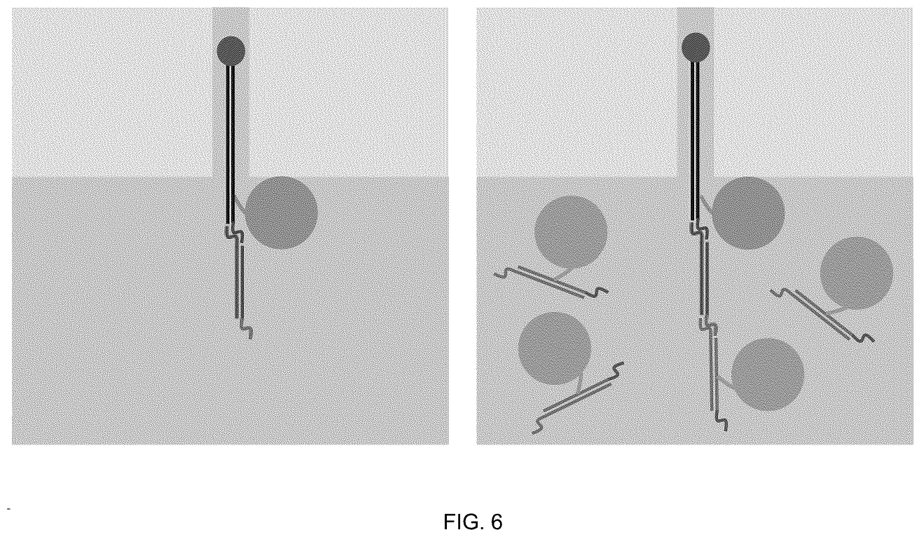

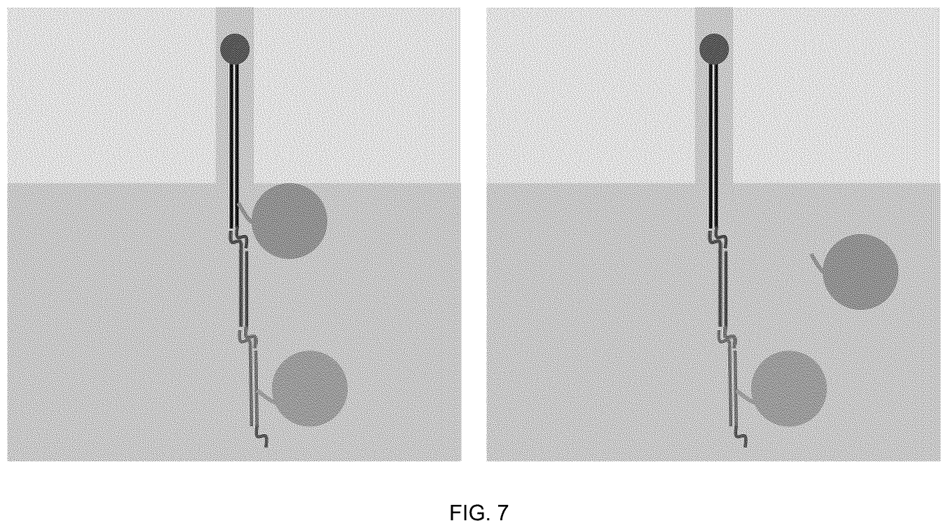

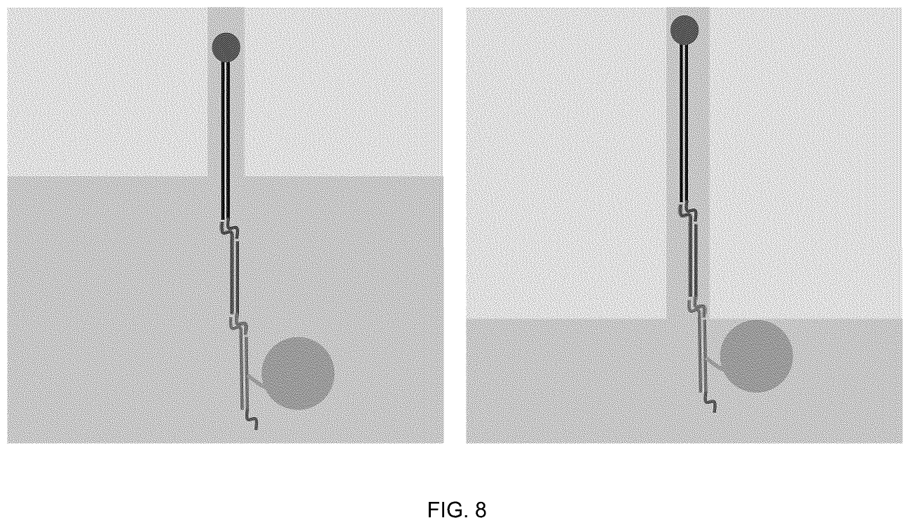

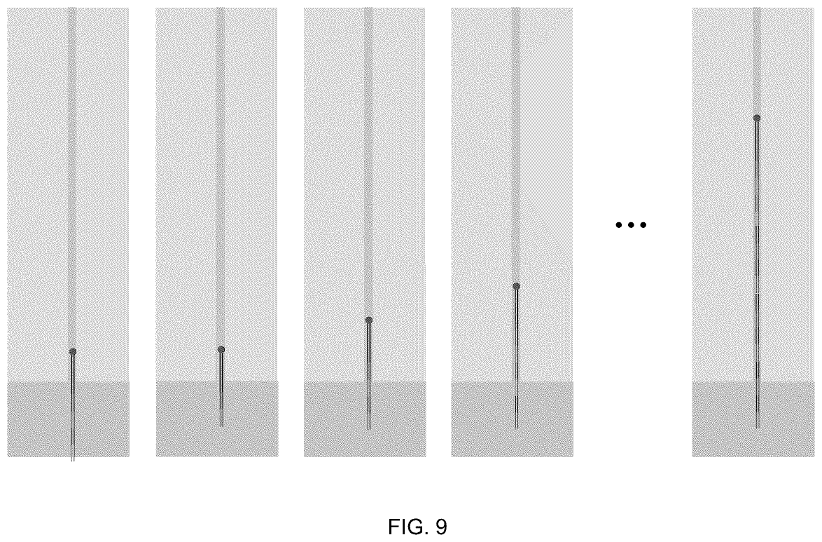

FIGS. 6-9 illustrate another preferred embodiment in which blocking groups are used to impose precision motion control on the growing strand. In this embodiment, as shown in FIG. 6 (left), there is again a point in the process where the shuttle with blocking group is staged in the nanochannel, and further segments may have been added as indicated. Then, a next segment is introduced via the microchannel, and this DNA segment has a second blocking group on it, as shown in FIG. 6 (right), but with an orthogonal reversible linker—i.e. having a cleavage chemistry that: (i) can be performed with without altering the first blocking group, and (ii) conversely, the cleavage chemistry of the first blocking group can be performed without altering the second blocking group. In certain embodiments, the block group may be the same in both cases, but with a different cleavable linker. Many admissible pairs of orthogonal cleavage chemistries are well known to those skilled in conjugation chemistry. For example, one linker could have a disulfide bond, cleavable by chemical treatment with DTT or TCEP, and the other could be a DNA oligo with Uracil bases in it, cleavable by digestion with uracil-DNA glycosylase (UDG) enzymes. Or dsDNA linkers could be used, with distinct and unique 20-based sequences in them (and not present in the target DNA being assembled) that are cleaved by cognately programmed CRISPR Cas enzymes. Or photo-cleavable and thermo-cleavable linkers can be paired, or paired with any of the above. As shown in FIG. 7 (left), after washing both cleavable blocking groups will be present on the strand, one actively stopping the motion again the inlet, and the other free in the microchannel. At this point, the first (active) blocking group can be cleaved, leaving the second group intact, as shown in FIG. 7 (right). After washing away the free group, the local configuration will be as in FIG. 8 (left), and now translation can be imposed on the strand without control, moving the strand into the channel, and as shown in FIG. 8 (right) the remaining blocking group sill now stop the motion at the precisely desired location that leaves the joinable end exposed to the channel ideally positioned for subsequent joins. This joining process can be repeated, alternating the use of the two orthogonal blocking groups to step along the motion discretely from one blocking group to the next, as indicated in FIG. 9.

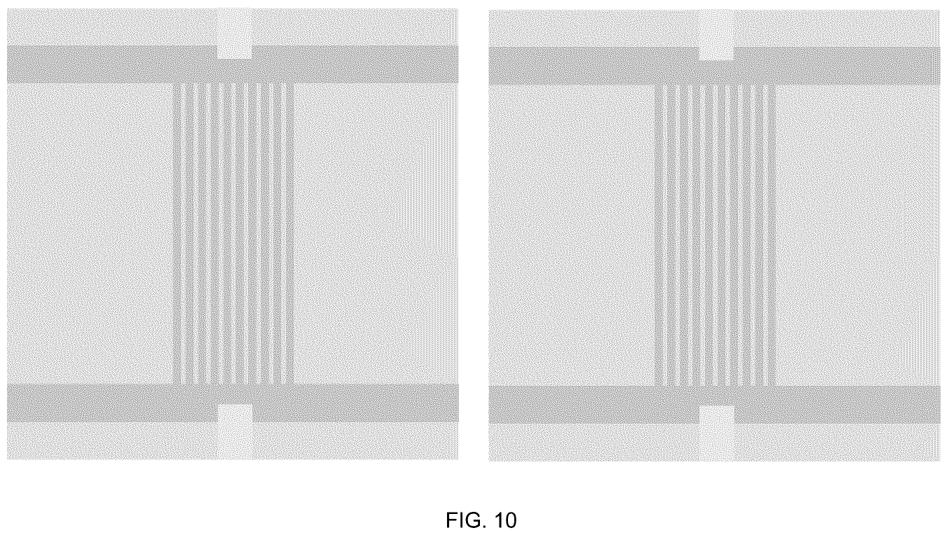

Using either of these blocking group motion control methods, for the production of multiple strands in parallel, as shown in FIG. 10 (right), the strands will remain synchronized by the frequent physical stops, even after many cycles of addition, whereas without such blocking groups to register the motion, the many strands would become de-synchronized over many cycles of addition as indicated for contrast in FIG. 10 (left).

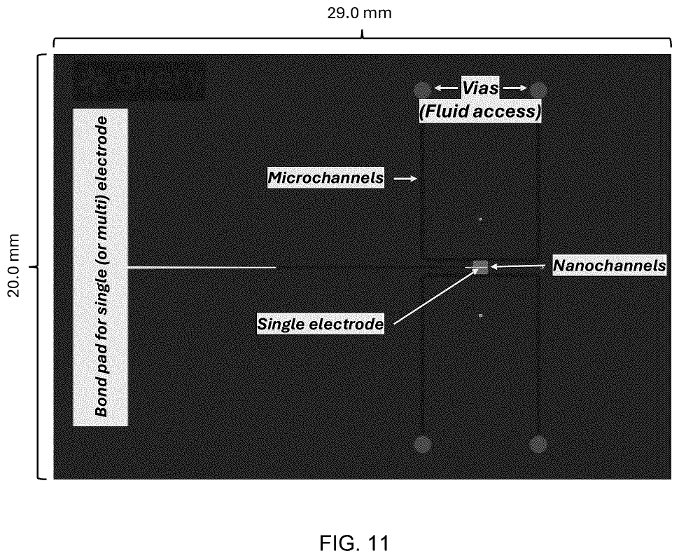

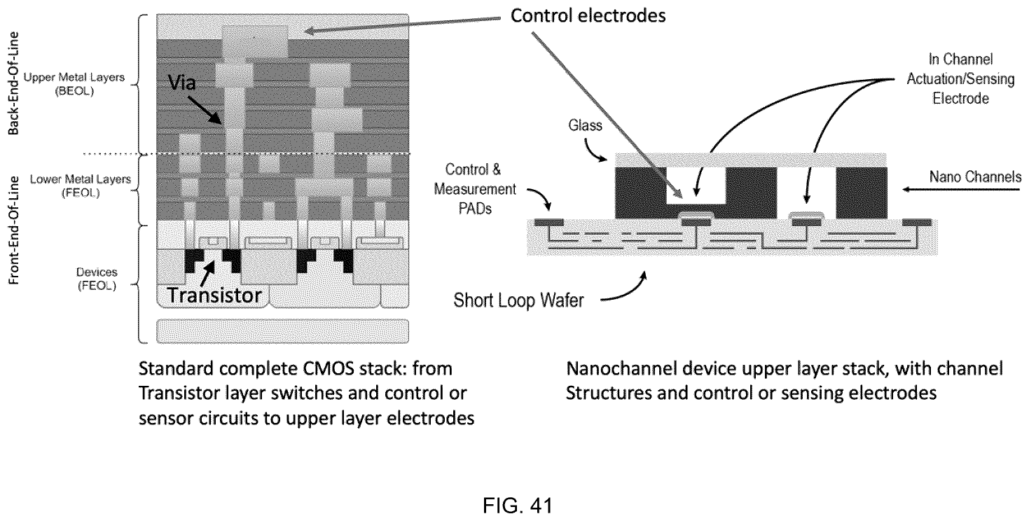

Certain embodiments of chips used for experimental demonstrations of this nanochannel assembly method are shown in FIGS. 11-15. These are passive devices (no integrated circuits, just leads that go off chip for connection to external voltage control circuitry), not CMOS chip devices. However, similar fabrication processes would be carried out by post-processing of CMOS chip devices in order to integrate nanochannels with CMOS chips, as indicated in FIG. 41, and it is apparent to those skilled in the art of nanofabrication how to carry out these processes on a CMOS chip substrate on which planar vias are exposed as sites for connection to the various control electrodes fabricated in this post processing, including the use of advanced photolithography patterning as an alternative to electron beam lithographic patterning. The specific passive chips shown here are made using tools at the Nano3 nanofabrication facility at UCSD and use a collection of semiconductor lithography methods to etch the structures into silicon wafers. This starts with a lithography mask design, as shown in FIG. 11. This pattern is used to define the geometry of the microchannels and nanochannels and electrodes for subsequent lithography steps.

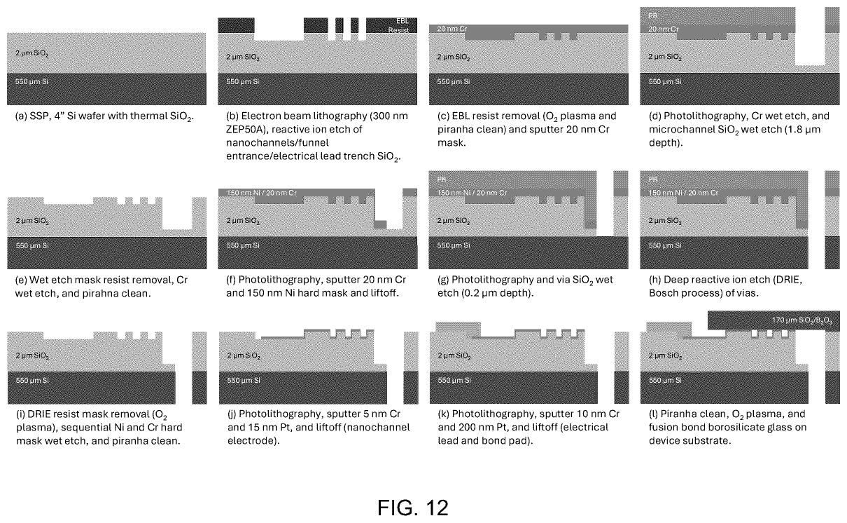

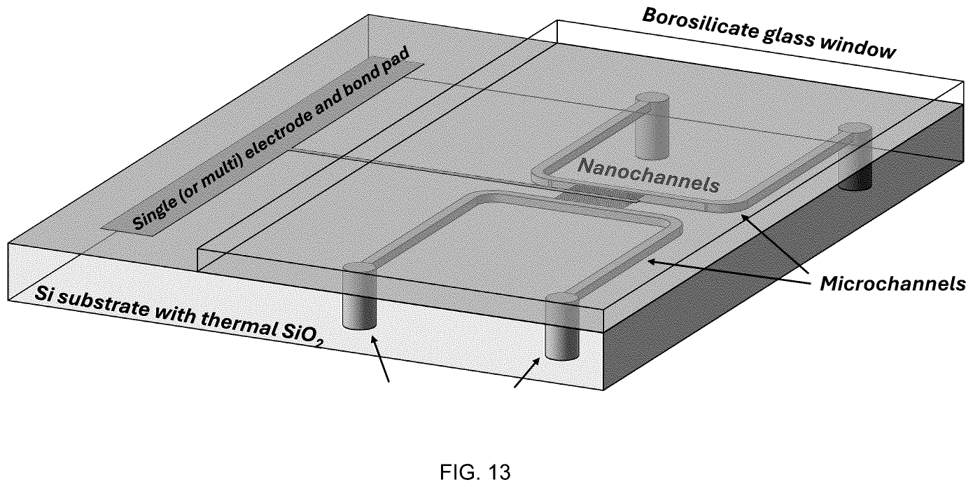

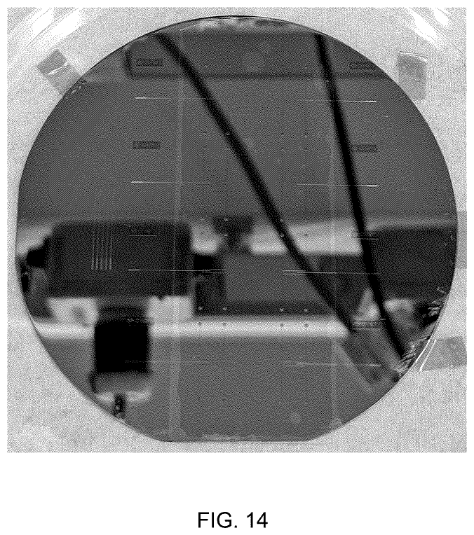

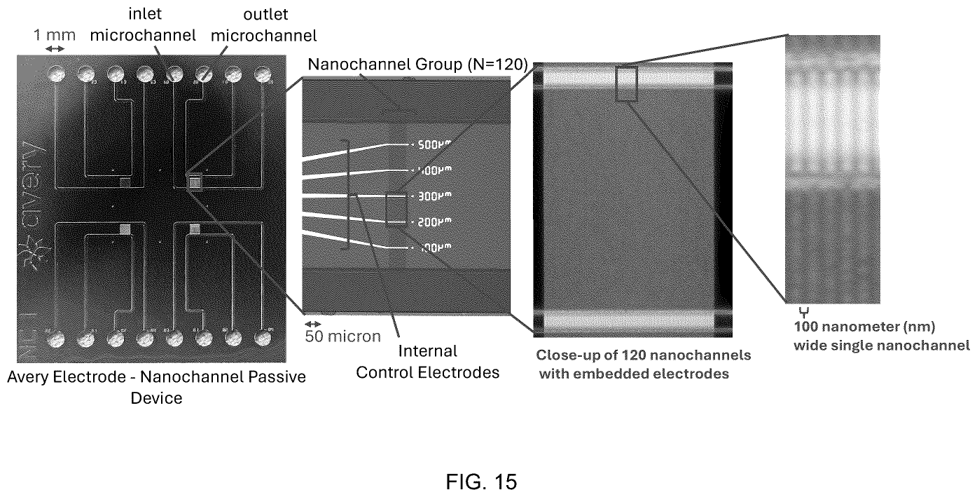

FIG. 12 shows an exemplary series of schematic lithographic processing steps used to make the channel and electrode structures on a silicon wafer. In this exemplary process, electron beam lithography is used to pattern the nanochannels in a resist, and after developing the resist, Reactive Ion Etching is used to etch the nanochannels. Then a photolithography resist is applied and the microchannels are patterned into this, and after developing these channels are formed via wet etching. Additional photolithography patterning, wet etching and deep reactive ion etching are used to make the deep wells (vias) that act as inlet and outlet reservoirs. In some embodiments, the inlet/outlet connects the nanochannels and microchannels. In some embodiments, the inlet/outlet is funnel-shaped. Finally, a layer of borosilicate glass is thermal fusion bonded to the top surface of the finished silicon substrates, in order to transparently seal the channels for fluidic control and imaging. In order to effectively attach this enclosing glass surface, it is beneficial that the upper surface of the nanochannel device be sufficiently planar. The main source of non-planarity is the embedded electrodes, as their thickness results in differing heights of the thickness-controlled layers deposited over them. One preferred embodiment that keeps the upper surface sufficiently planar is to keep the thickness of these electrodes less than 100 nanometers, and preferably less than 50 nanometers, and most ideally in the range of 10 to 30 nanometers. Here these were fabricated with a 50 nm thickness. In other preferred embodiments, this planarity can be achieved by using Chemical Mechanical Polishing (CMP), which is a routinely used method in semiconductor chip manufacturing. The resulting devices are shown in a 3-D geometry view in FIG. 13. The nanochannels are in bundle of replicates that extend between inlet and outlet microchannels, with reservoirs (vias) for external fluid inlets and outlets. A single internal control electrode is also shown. FIG. 14 shows a finished silicon wafer of such devices, with 6 devices residing on the 4-inch wafer. As depicted in FIG. 14, a batch of 8 nanochannel device chips are on a 4-inch wafer after fabrication and borosilicate glass window bonding. FIG. 15 shows the details of one such finished device: At left is the full chip, which has 4 independent nanochannel devices on it, each having inlet and outlet microchannels and a nanochannel zone with 120 parallel nanochannels spanning the microchannels. The first inset image (second from left) shows a close-up of the nanochannel zone, showing 5 embedded internal gold electrodes that can be used for electronic position control of DNA in the channel, as in FIG. 2c. The next inset, third from left, shows a further zooming in on the 120 nanochannels, and the final inset, right, zooms in to show the individual channels which are 100 nm wide and deep, and separated from each other by 100 nm wide walls.

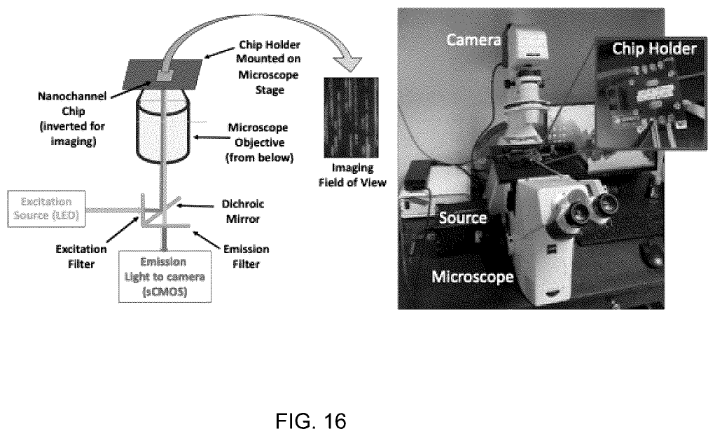

FIG. 16 shows an experimental station used for doing experiments on the nanochannel chips. The chip is mounted within a flow cell and connected to a fluidic pump and electronic power supply, to control the fluidics and channel electrodes. This flow cell is mounted in a high-magnification fluorescent microscope in order to image the DNA in the microchannels and nanochannels. A schematic of the imaging system is shown at left, including an exemplar image of green stained DNA residing in the nanochannels.

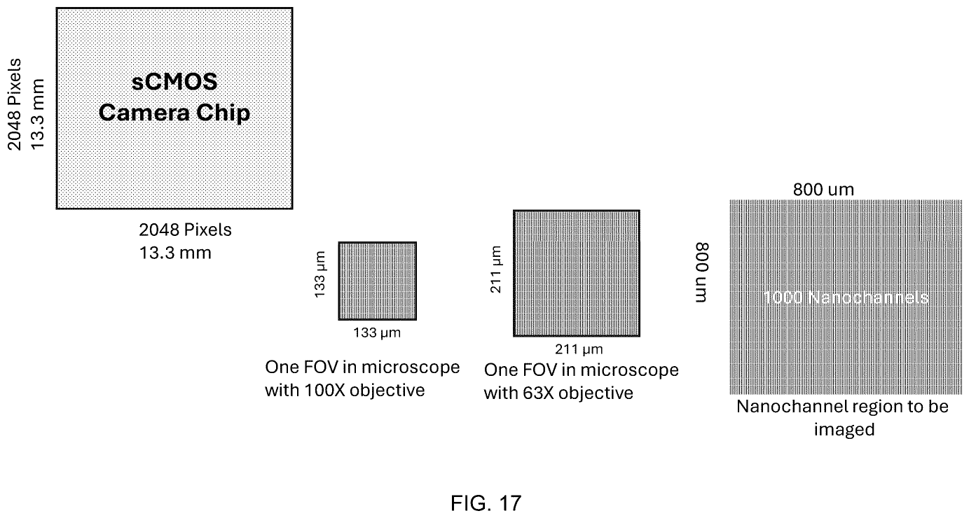

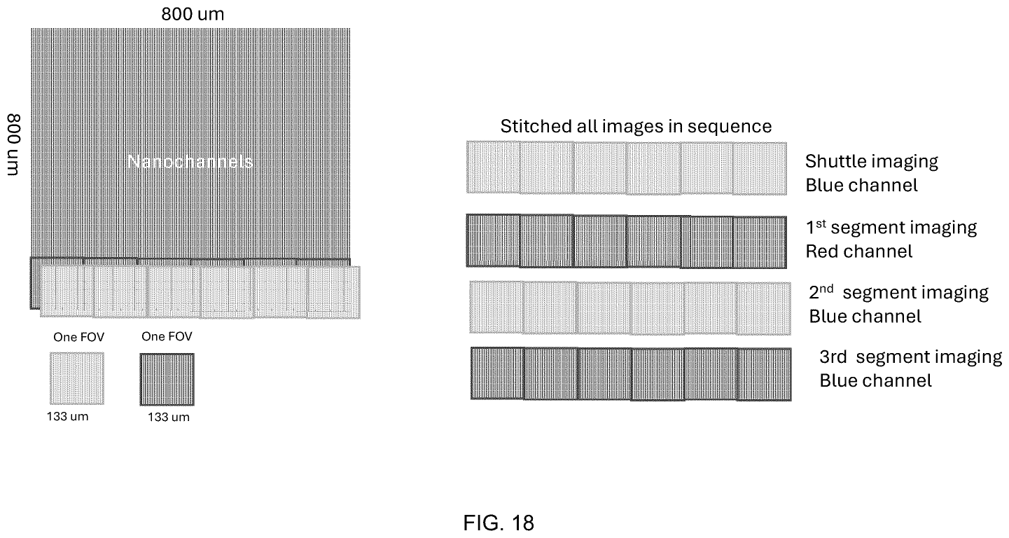

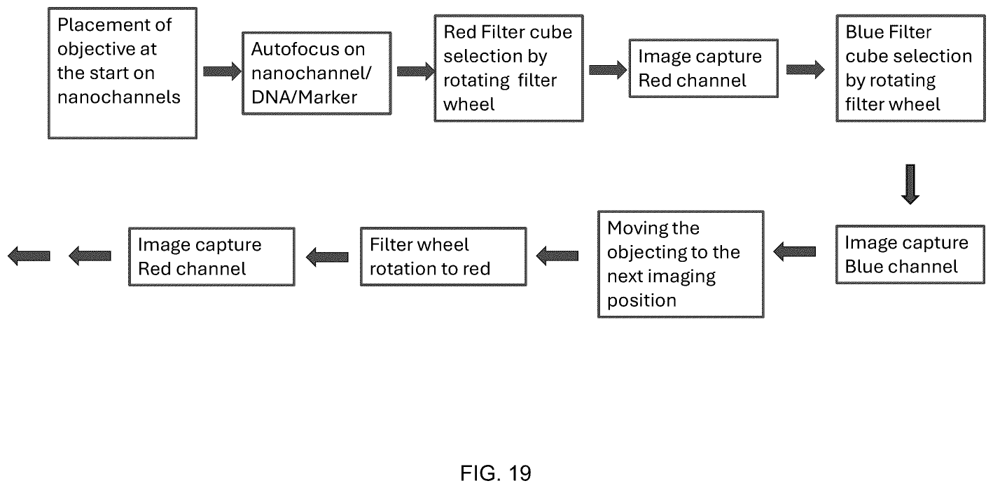

FIG. 17 illustrates the imaging geometry of this system: The scientific sCMOS camera has 2048×2048 pixels on a 13 mm×13 mm physical imaging array. Depending on the magnification used on the microscope (100× or 63×) this can image only various partial portions of a target nanochannel region, as shown. The Field of View (FOV) in the microscope with a 1× objective =13.3 mm×13.3 mm, the FOV with 100× objective=(13.3 mm/100)×(13.3 mm/100)=133 μm×133 μm, and the FOV with 63X objective=(13.3 mm/63)×(13.3 mm/63) =211 μm×211 μm. Thus, as indicated in FIG. 18, to image an entire set of nanochannels on one device, in both red and green fluorescent channels, it requires scanning-imaging across the desired imaging area, as well as changing between red and green filters. This is done using the programmable microscope controller to automate this process, with the automation sequence as shown schematically in FIG. 19. As shown as an example of an image processing modality using multiple FOV scanning in FIG. 18, the total number of nanochannels is about 1000, the Pitch is 600 nm+200 nm=800 nm, and the total length of the nanochannel=800 nm×1000=800 μm. One field of view with 100X objective can image 133 μm×133 μm. To image the whole nanochannel entrance of about 6 imaging positions, 2-color imaging will require capturing 12 images.

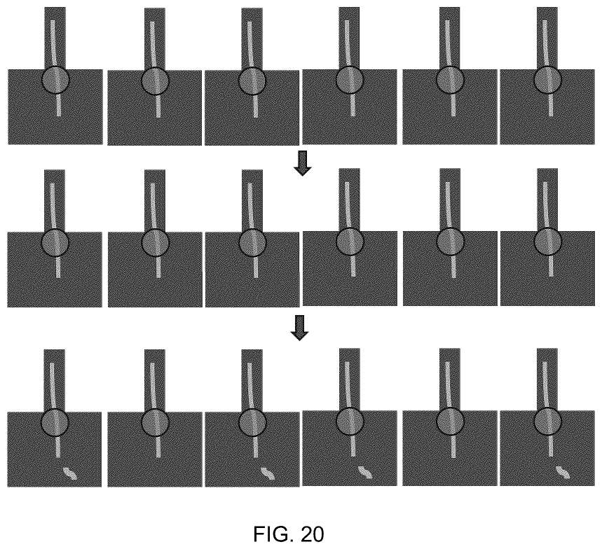

FIG. 20 illustrates conceptually how the red and green fluorescent images can be used to observe the serial addition of pieces in the nanochannel, starting with the shuttle loading, by observing in real-time the alternating green and red labels on successive incoming pieces. If the correct ordering of light color in time is not seen at the channel entrance, it is interpreted as showing that incorrect assembly has occurred. As an example, the Green Channel Imaging only shuttles in nanochannel entrance. Overlay of Green and Red Channel Imaging: After the joining of first (red labeled) segment, a defective join is detected. Overlay of Green & Red Channel Imaging: After the hybridization of second (green labeled) segment, defective joins are detected.

Shuttle Construction: The Shuttle is essentially a long DNA body with a cleavable blocking group and an exposed joinable DNA end. To those skilled in the art of molecular biology, there are many ways to design and to build such a molecule, and all such obvious variations are intended to be encompassed by this disclosure. Provided herein are several preferred embodiments of the construct and production protocols for the purpose of illustration or reduction to practice, and this is not meant to restrict the form of the shuttle or methods of fabrication disclosed herein.

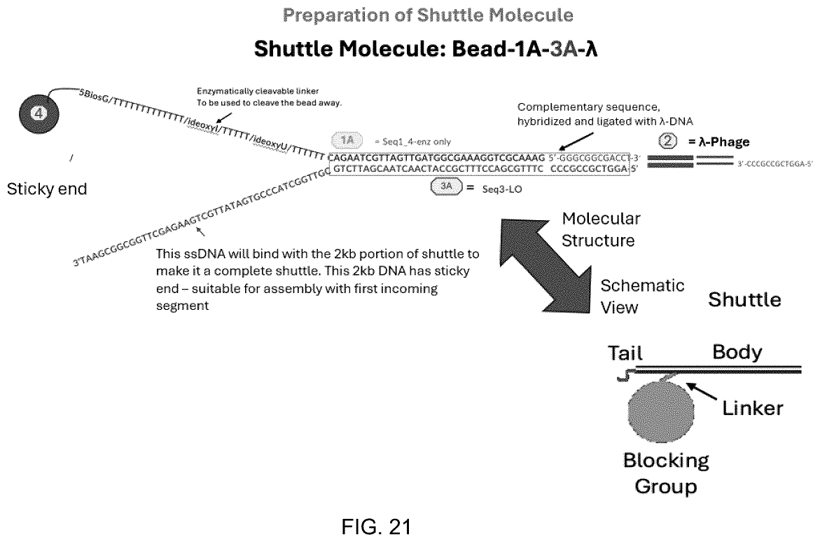

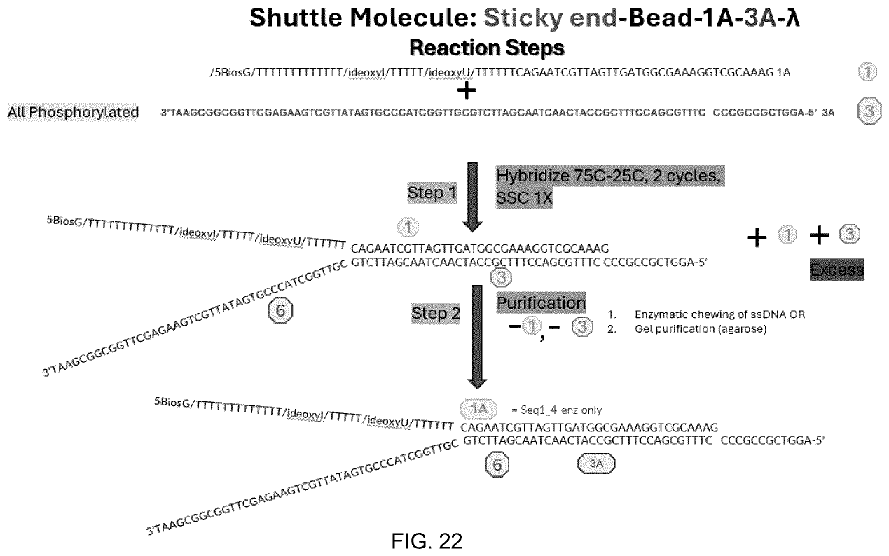

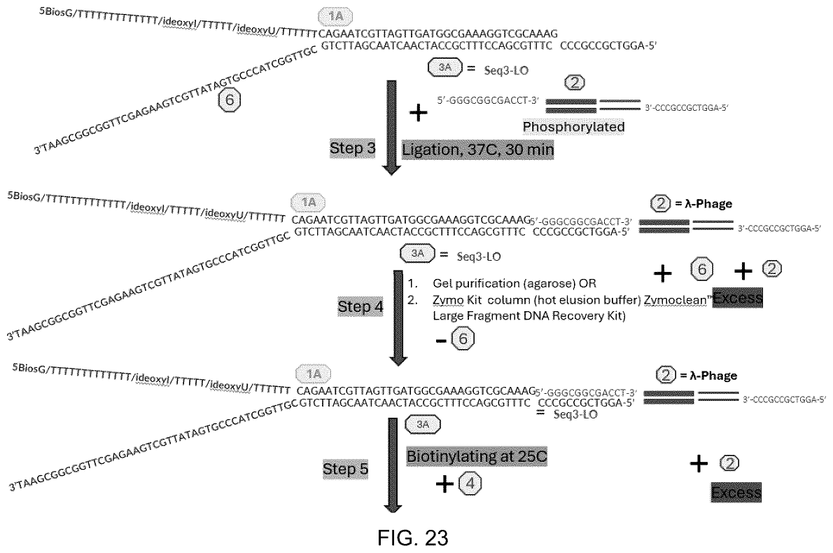

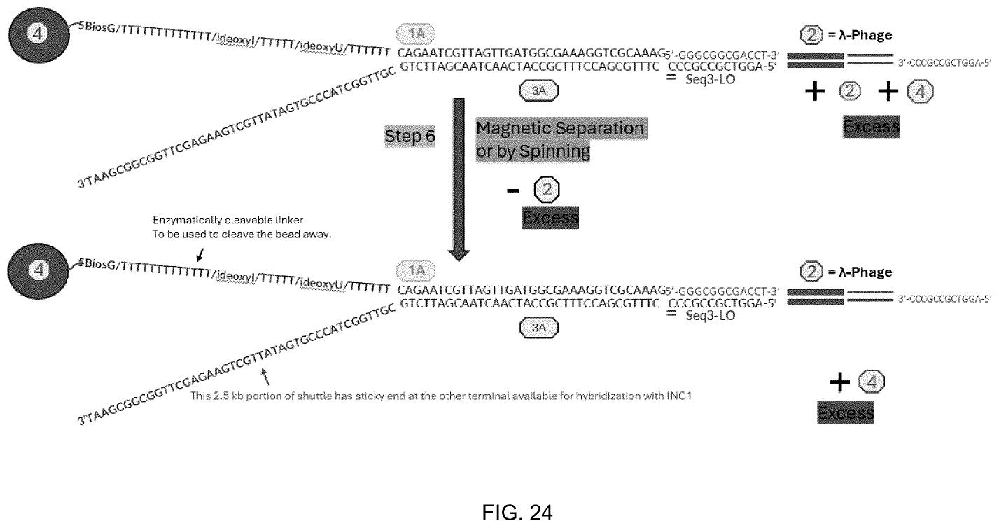

Shuttle V1 Format and Preparation Protocol: The model Shuttle form here is shown in FIG. 21, which highlights the comparison between the cartoon schematic of the Shuttle (lower right) and the precise molecular form (upper left). Here the form is a 48 kb long Lambda phage dsDNA (approximately 15 microns long, extended) that is attached to sequences on one end (designated 1A and 3A) that form a double-stranded segment that is ligated to Lamda, and that have on the other end one strand linked to a streptavidin-coated bead several hundred nm in diameter, via a biotin linker on the termini, and the other strand free to hybridize to an incoming segment via a universal sequence used to initiate the growing strand. Thus, the full molecular construct is denoted Bead-1A-3A-Lambda. The bead used in this example is a 350 nm diameter streptavidin coated polystyrene bead. The major steps of the construction of this shuttle molecule are shown in FIGS. 22-24. These major steps are as follows:

Before use, all oligos and lambda DNA was phosphorylated with T4 polynucleotide kinase (PNK) in PNK buffer in presence of ATP (all reagents from NEB).
Step 1: Hybridization of equimolecular mixture of 1A and 3A (both phosphorylated) was done by thermocycling two times between 75C and 25 C in 1X SSC buffer.
Step 2: 4% agarose Gel purification of the hybridized molecule (construct 6) to remove free 1A and 3A. Then cleaned the hybridized molecule 6 by Zymo kit.
Step 3: Equimolecular mixture of hybridized DNA construct 6 and Lambda DNA (phosphorylated) from NEB was hybridized and ligated with HiT4 ligase for 30 min at 35C and then enzyme was deactivated at 65C for 10 minutes.
Step 4: Excess hybrid construct 6 was removed by gel purification (agarose) and the sample from the wells of the gel was collected. Zymo Kit column (hot elusion buffer) Zymoclean™ (Large Fragment DNA Recovery Kit) was used for purifying the mixture of 6-Lambda construct and free Lambda.
Step 5: Now streptavidin coated polystyrene beads (average size 350 nm, Spherotech, IL) or streptavidin coated magnetic beads (550 nm, Luna Nanotech, Canada) was added to equimolecular amount of 6-Lambda (theoretical) with free lambda in excess in it.
Step 6: The Shuttle molecule was separated from free lambda by spinning at 1500 rpm of for 20 min of by using magnetic bar. The final sample contains Shuttle constructs and free beads.

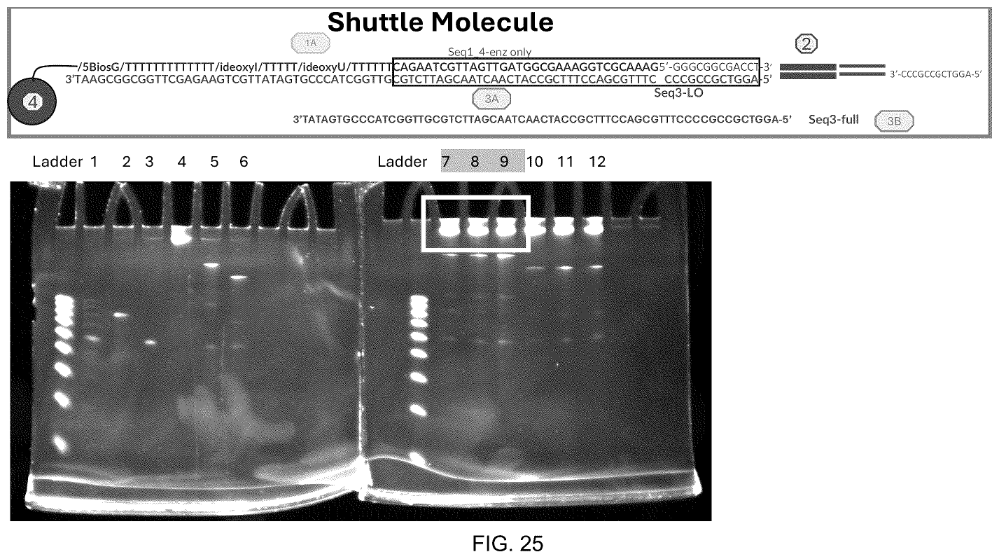

FIG. 25 shows a gel analysis of the shuttle construct. The various lanes show portions of the construct for reference, but lanes 7, 8, 9 show the full Bead-1A-3A-Lambda construct. The properly formed shuttle is large and trapped near the top of the gel in these lanes (highlighted). Information about the lanes of the gel is as follows:
Ladder: 100-20 bases oligos; 1=1A=61 bases oligo; 2=3A=88 bases oligo; 3=3B=65 bases oligo; 4=Lambda; 5=1A-3A hybridization length=35 bases MT of hyb length=80 C; 6=1A-3B hybridization length=35 bases; 7=1A-3A-λ, hybridization length=47 bases; 8=1A-3A-λ, hybridization length=47 bases; 9=1A-3A-λ, hybridization length=47 bases; 10=1A-3B-λ hybridization length=47 bases; 11=1A-3B-λ hybridization length=47 base; 12=1A-3B-λ hybridization length=47 bases.

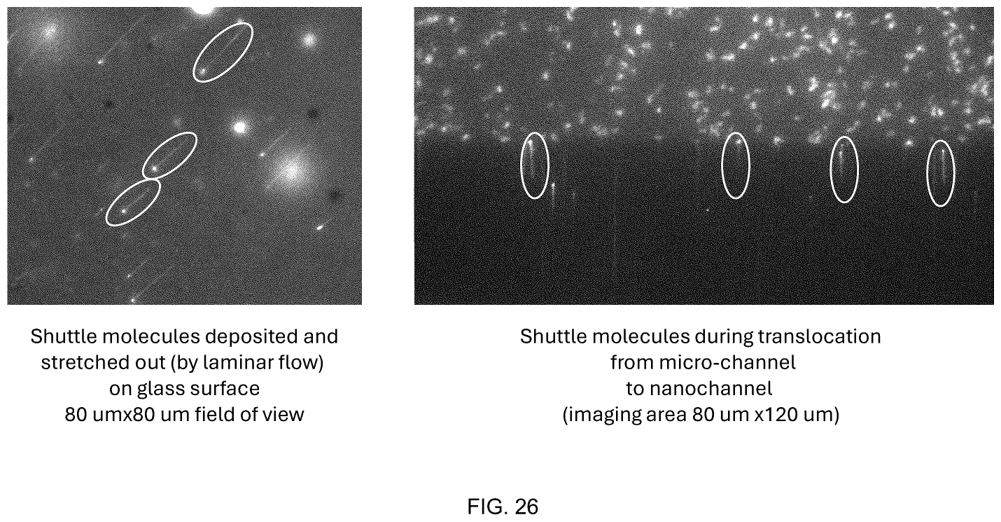

FIG. 26 shows single molecule imaging of the constructed Shuttle molecules. At left are Shuttles deposited on a glass slide under laminar flow conditions so that they stretch out on the surface. Molecules are fluorescently stained, and 3 molecules are highlighted (encircled), showing the bright bead at one end, and the long Lamda tail extending from the bead. At right are the Shuttles actually loaded into nanochannels on the nanochannel device; the bright spots on the upper half are free shuttle DNA in the nanochannel, and some Shuttles have been captured into nanochannels, where they are staged at the entrance with the Lambda body extending into the channel, and the bead trapped at the nanochannel entrance. This is the critical priming and staging step of the assembly process.

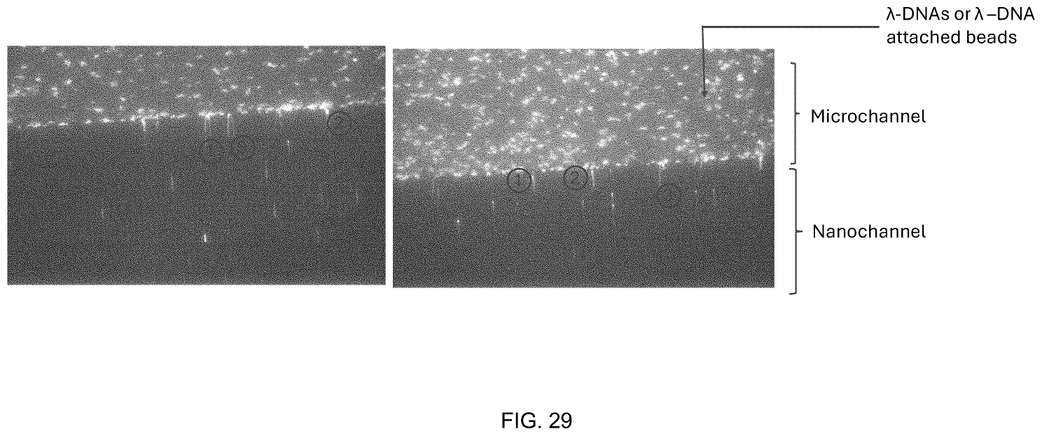

FIG. 29 also shows the loading and staging of many Shuttle molecules at the nanopore entrances (the entrance of nanochannels). Imaging Protocol nanochannels: all the DNA (4 ng/ml) samples were stained with intercalating dye YOYO-1 iodide (Thermo Fisher) (1 molecule/10 bp) in presence of 0.4 M DTT (Sigma). The sample was diluted by two times using the flow buffer consisting of 1 TBE, 0.5% Tween. For all the solution preparation ultrapure distilled water was used. (Sigma). DNA molecules are electrokinetically driven at 5-10V at the port of entrance of the chip and allowed to populate at the nanochannel entrance for 2-3 min. Applying higher voltage (˜20V) the populated molecules are moved into nanochannel structure 200×200 nm of the chip to transform the compact globular DNA structure to an open linearized structure. At the 200 nm channel area the molecules adopt relaxed linear form with some heterogeneity on the backbone. FIG. 29 shows an image for DNA molecules at equilibrium in a nanochannel. A buffer consisting of 0.5 TBE, 1.8% Tween, 5% PVP have been used to flow the DNA molecules resulting in a stretch of 65% of 0.34 nm/bp.

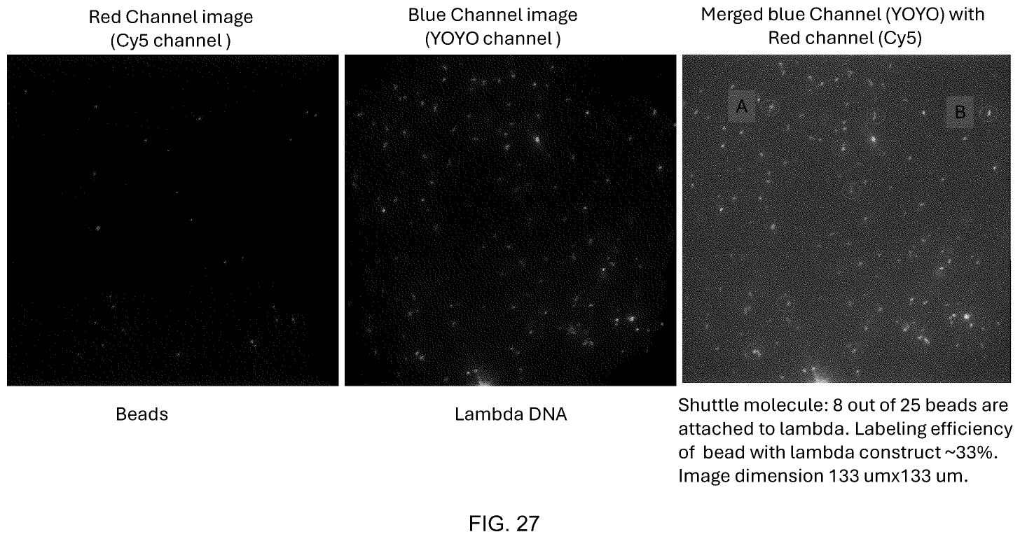

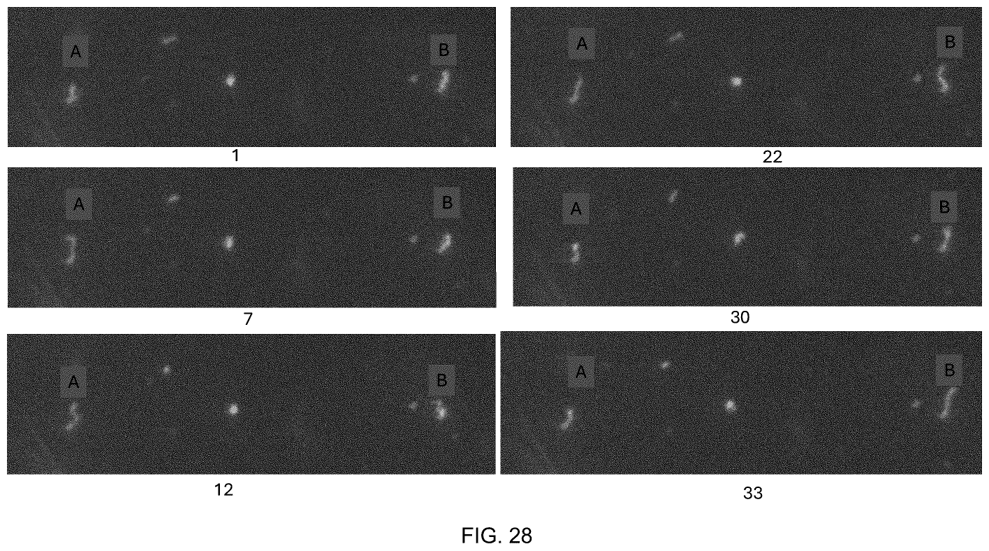

FIGS. 27 and 28 show 2-color red/green fluorescent imaging on single molecule Shuttles deposited on a glass slide. Hear the bead is stained red (Cy5 dye), and the Lambda DNA body is stained green (YOYO stain). FIG. 27 shows the single channel images at left, and the false color red-green overlay at right, which makes the Shuttles visible as a red head with a long green body. FIG. 28 shows 6 frames from a video of these in the laid out under a coverslip in solution, where the motion of the Lambda and the time sequence make it clearer there is a stationary red head and a long green tail. Imaging protocol: The Shuttle construct was stained with YOYO intercalating dye and imaged after dilution and at the single molecule level placing in between two glass coverslips. Images were collected first by 647 nm excitation light and then by 488 nm light with correct dichroic and emission filters in place and images were overlaid using Image J. Microscopy and image processing used for the single molecule images: The imaging was done in epi-fluorescence mode using Microscope: Zeiss: Axio Observer Z1 Inverted Phase Contrast Motorized and Plan-Apochromat 100x/1.4 Oil M27 Thread Size: 420790-9901 objective. YOYO-1 iodide (491 nm, absorption; 509 nm, emission), the DNA backbone staining dye was excited using 488 nm (Zeiss Calibri 5 light source) whereas Alexa 647/Cy3 (650 nm, absorption; 665 nm emission) 633 nm LED light (Zeiss Calibri 5) was used for excitation (Zeiss). We used a filter cube consisting of single band dichroic and single band pass emission filters Filter: 49002-ET-EGFP (FITC/Cy2) ET470/40x, 25 mm in a ring T4951pxr, 25.5×36×1 mm ET525/50m, 25 mm in a ring (Chroma Technology Corp., Rockingham, VT, USA) for detection of YOYO-1. For detection of Alexa 647, the single band filter cube filter: 49009-ET-Cy5 Narrow Excitation ET640/30x, 25 mm in a ring T6601pxr, 25.5×36×1 mm ET690/50m, 25 mm in a ring has been used. The emission signal was detected by a: Teledyne Photometrics-PRIME-BSI-R-M-16-C-PCI-E Prime BSI back-thinned 4.2MP sCMOS microscope camera with PCI-E connection. The data were recorded at 100 ms exposure time using Micromanager software. All the image analyses were done using Image J (National Institutes of Health, USA).

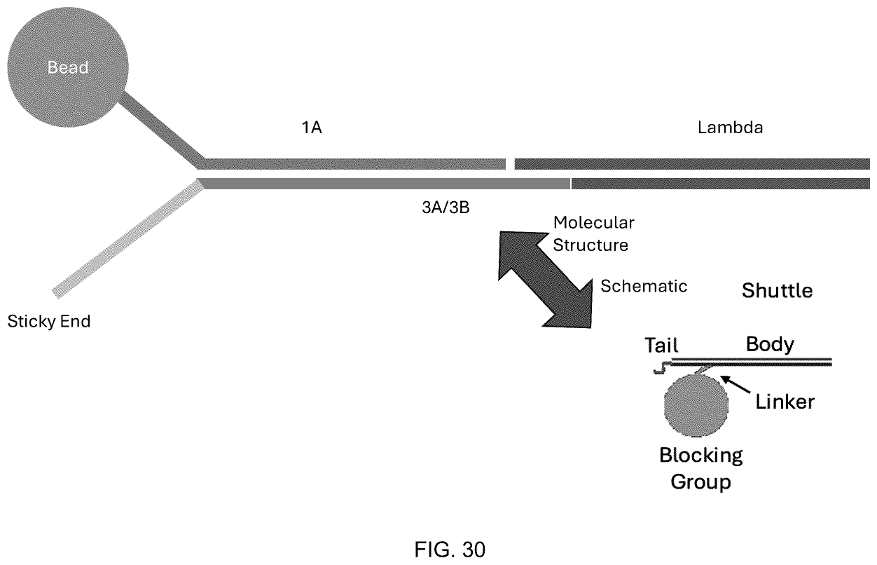

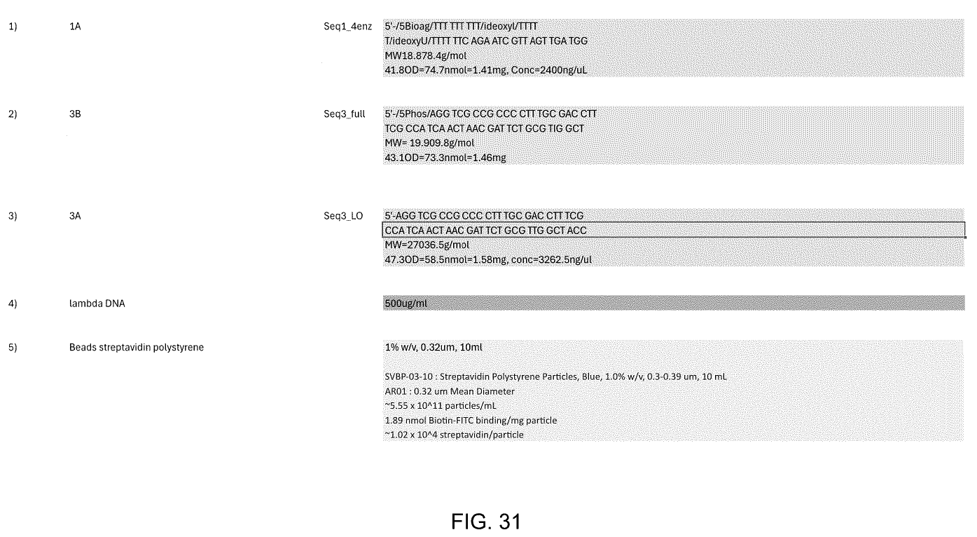

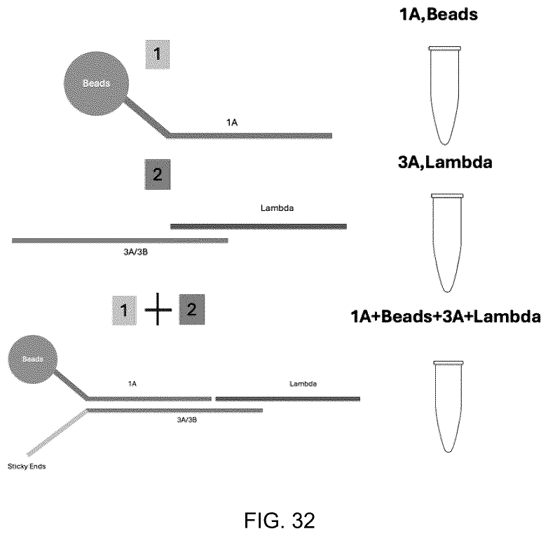

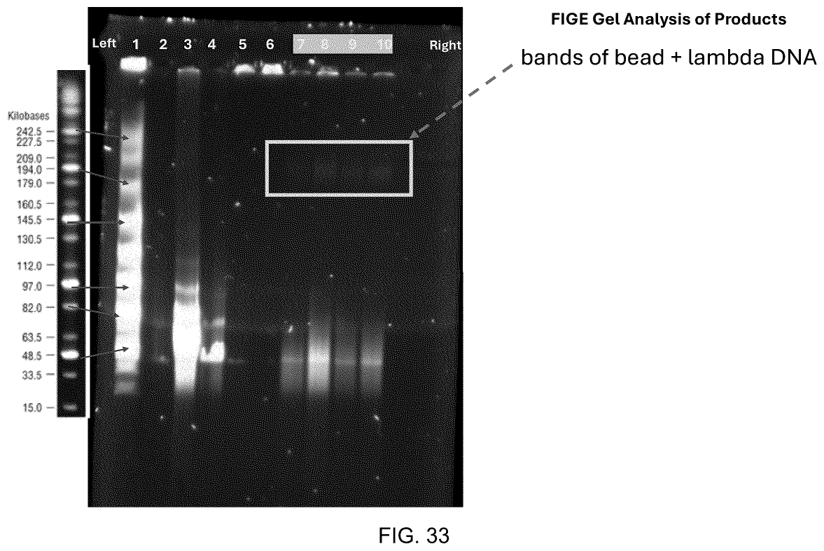

Shuttle V2 Format and Preparation Protocol: FIG. 30 shows another model architecture for the Shuttle, similar to that of FIG. 21, where the body is Lambda phage DNA, joined to a segment made from strands 1A and 3A or 3B, and with one end conjugated to a bed, the other free as a universal sticky end. The table in FIG. 31 gives the precise details on these DNA components and the beads used. FIG. 32 illustrates a different preparation protocol, which joins 1A the bead in one reaction, 3A/B to Lambda in another reaction, and then reacts these products together: This stepwise assembly of shuttle DNA-bead construct improves the yield of fully formed constructs. FIG. 33 shows a FIGE gel analysis of the products, which is a type of time-varying field gel capable of resolving very large constructs such as the shuttle; highlighted in lanes 7/8/9/10 are bands that represent the desired well-formed products. Information on the lanes of the gel are as follows, from left to right: 1) Ladder; 2) Empty well; 3) Lambda DNA only (2 μl loading volume); 4) Lambda DNA only (4 μl loading volume); 5) Bead only (2 μl loading volume); 6) Bead only (4 μl loading volume); 7) Bead+lambda, old prep (2 μl loading volume); 8) Bead+lambda, old prep (4 μl loading volume); 9) Bead+lambda, new prep (2 μl loading volume); 10) Bead+lambda, new prep (4 μl loading volume).

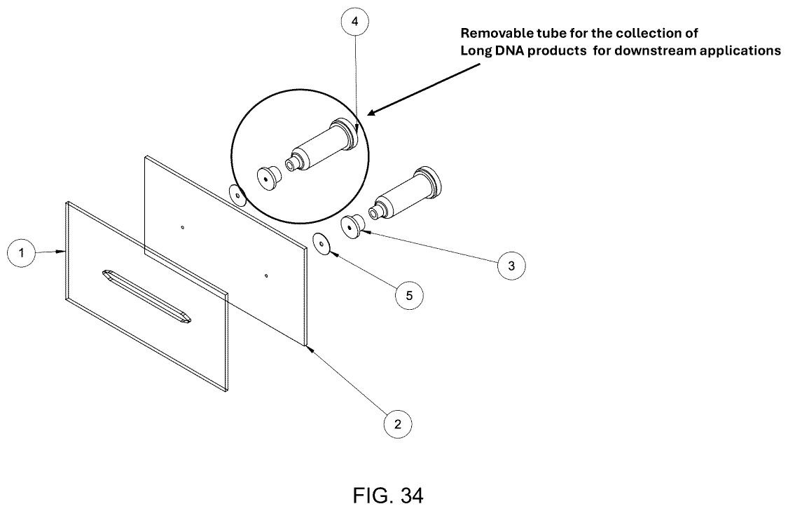

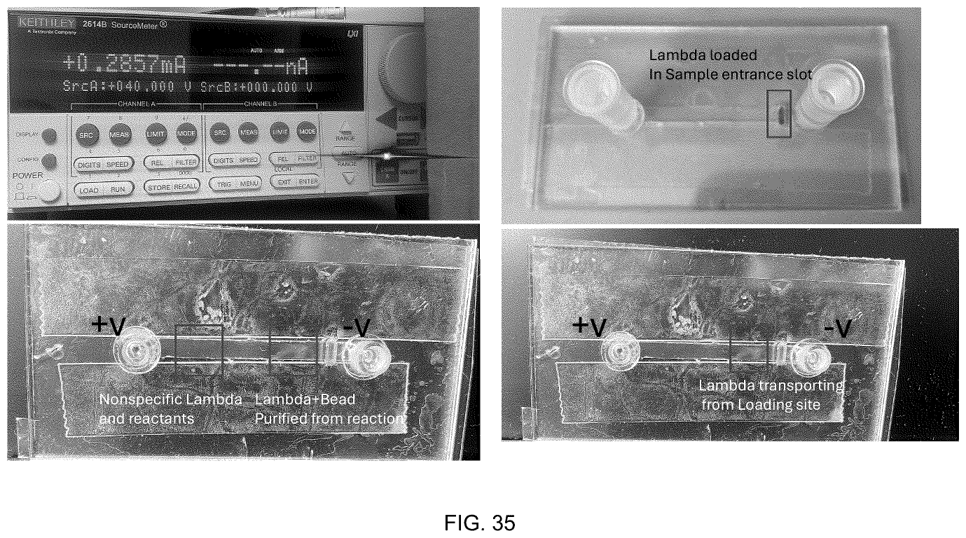

Collection of Long Assembly Products: FIG. 34 and FIG. 35 show means of collecting and transporting the materials produced by the long assembly process. As indicated in FIG. 2f, the long DNA products need to be deposited in a thin agarose gel medium for storage and transport for other downstream uses, to avoid breakage. FIG. 34 shows the schematic for a simple device that can electrokinetically move the long DNA constructs from the chip exit and selectively into a storage/transport tube loaded with agarose. The device defines a transport channel by the two bottom plates and has collection tubes at each end. It is filled with agarose, and DNA is transported through it electrokinetically, under the control of external electrodes that interface to the device. FIG. 35 shows the device in operation, where it is used to transport and capture Lambda DNA as a demonstration, showing transport and collection of samples from long assembly. The λ-DNA sample is loaded into designated microwell slots on the chip and driven into a low-melt agarose gel matrix using an applied electric field. The DNA migrates through the gel and accumulates in a downstream reservoir, enabling stable storage and retrieval for subsequent downstream analysis. In the example shown lambda is loaded in (upper right) and transported through the gel channel (lower right), while in the lower left the image the device is used to purify Lambda+bead conjugates from bare Lambda, as a model for purifying the shuttle constructs from the reaction ingredients used to make the shuttle. In the upper left is show the power supply which provides the driving voltages for the electrophoresis/electrokinetics.

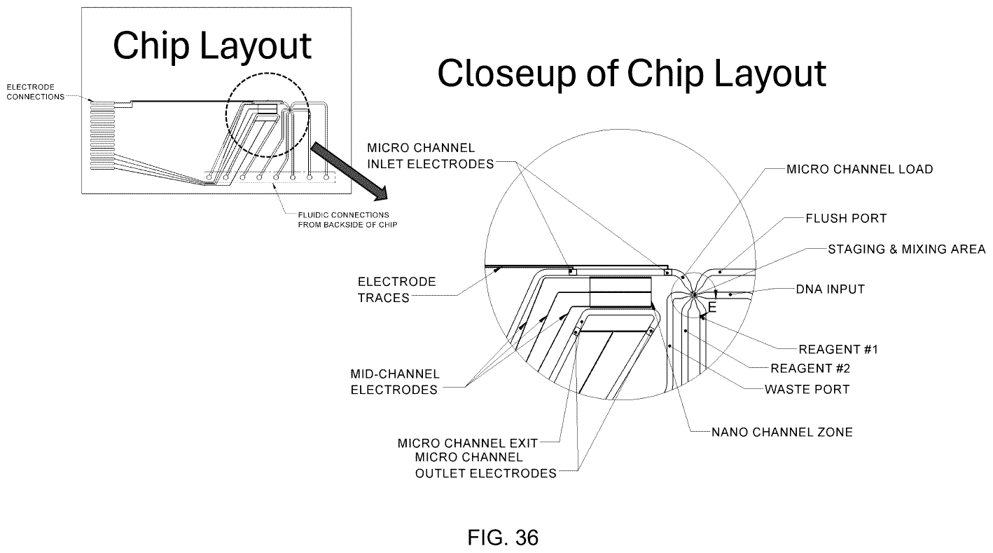

Experimental System Architecture Embodiments: FIGS. 36-40 show the design of the experimental rig that is used to fully automate the basic assembly workflow for performing assembly on the passive chip devices.

FIG. 36 shows the chip layout for a preferred embodiment of the experimental platform for the passive chip device. At upper left is a high-level view of the entire nanochannel MEMS chip, and at lower right is a blown up inset showing details near the nanochannels. The passive chip contains larger microchannels which deliver bulk reagents and DNA fragments into the nanochannels. Reagents are fed into the chip via ports on the underside of the chip and are joined at a staging and mixing area prior to being injected into the micro channels for exposure to nanochannels. Output microchannels are used to carry away material passing through the nanochannels and to provide electrical potential across the nanochannels. Electrodes are placed in both the microchannels and nanochannels to aid in DNA movement.

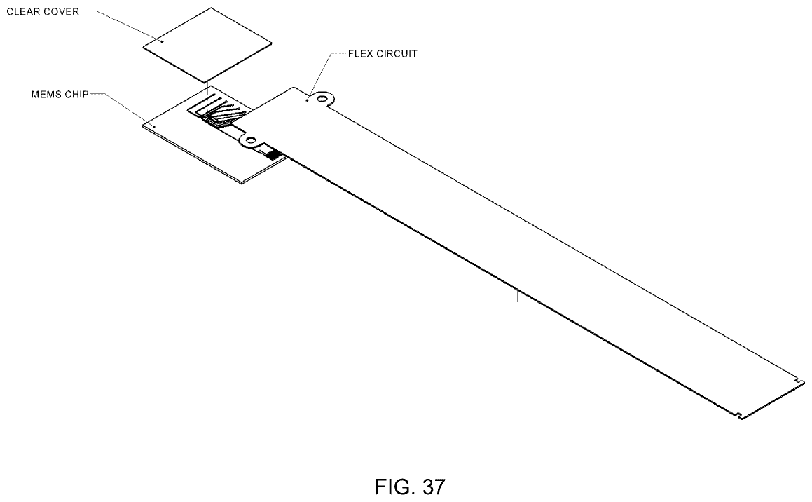

FIG. 37 shows an exploded view of the chip holder assembly, showing the chip assembly including the nanochannel MEMS chip with a bonded glass cover sealed over the etched channels. Electrical connections are made to the chip using a flex circuit which is bonded to the chip with a conductive epoxy film adhesive bond Anisotropic Conductive Film (ACF).

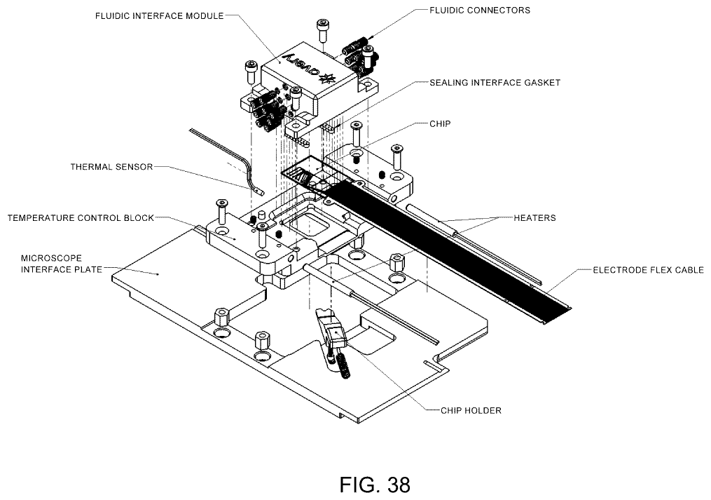

FIG. 38 shows the entire chip holder which facilitates holding the chip in the microscope stage, provides thermal management of the chip for reactions which require incubation temperatures and provides electric and fluid connections to the chip.

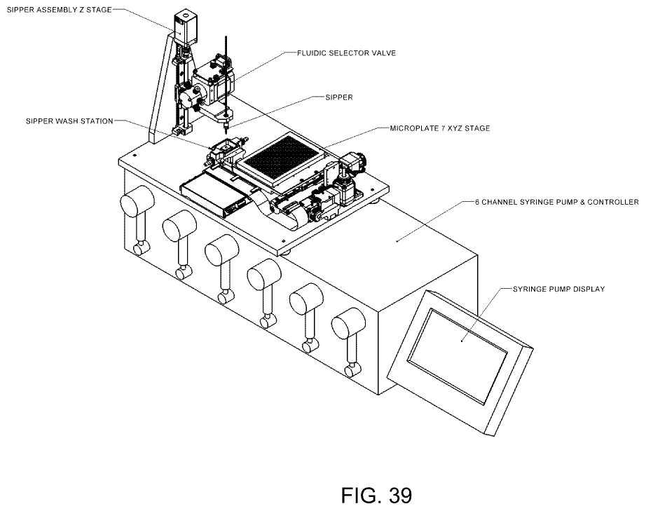

FIG. 39 shows the front-end stage to the rig. This is a Fluid Sampler and Delivery Assembly that controls all fluidic delivery to and from the chip. A 6-channel, computer-controlled syringe pump is connected to the chip via the port connections. A rotary fluid selector valve allows the drawing of DNA fragments from a sipper inserted into a microplate and loads the sample into the microchannels. The assembly also includes a wash station to clean the sipper between samples to prevent contamination.

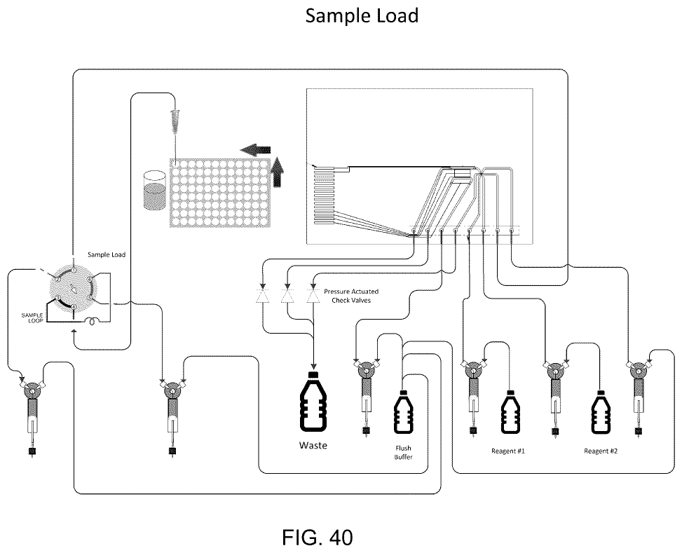

FIG. 40 shows an exemplary autosampler fluidics input schematic with a sample load. The fluidic logical architecture for the autosampler is shown, which loads sample from a 96 or 384 microplate well into the sample loop on 6-port rotary valve that supports the loading of the shuttle and the fragment library, as well as the washing between samples and other processes such as cleavage of the blocking groups. Any number of input fragments can be handled by manually swapping in well plates as needed to access all fragments.

CMOS Chip Device Embodiments: An ideal format for the nanochannel device is a CMOS chip device, so that control and sensing electrodes can be integrated into the device in high numbers and at high density, to support many parallel replicated nanochannels on chip, and multiple non-replicated instances on one device. In addition, modern advanced photolithography tools available at CMOS foundries have the resolution and precision to directly fabricate the top layer nanochannels themselves. FIG. 41 shows a first exemplary scalable chip device, showing making channels-on-CMOS. Shown at left is the standard layer stack for a CMOS chip device. The transistors exist at the lowest layer (Front-End-of-the-Line, FEOL) and the layers of wiring for building circuits exist in the upper layers (Back-End-of-the-Line, BEOL). Surface electrodes are produced in CMOS by using vertical wiring (vias) and horizontal wiring (interconnects) to form a lead from the circuit layer to electrodes fashioned as segments of interconnect or as contact pads. At right: nanochannels can be fabricated in the upper layers of silicon dioxide on a CMOS chip, using advanced lithography tools available at standard CMOS foundries, and the control electrodes are formed from the BEOL metal stack, with leads running down to the transistor circuitry using multiple BEOL and FEOL via (vertical) connections. The development of these structures can be done in “short-loop-wafer”, which are agile, rapid prototyping partial material stacks available from the foundry.

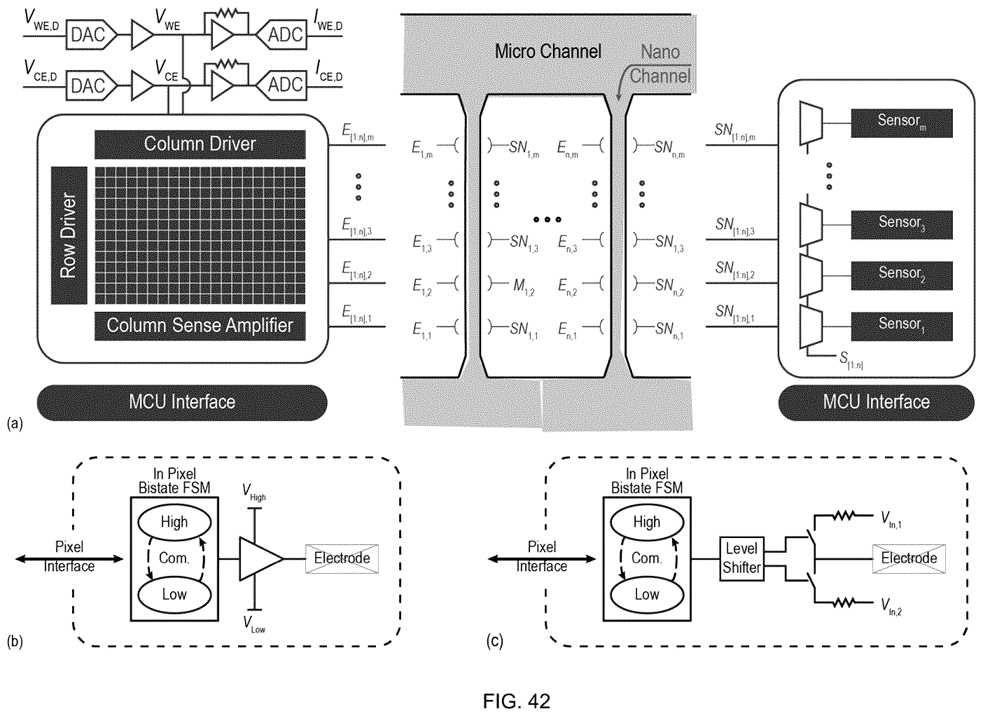

FIG. 42 shows a second exemplary scalable chip device, showing a CMOS chip device. Shown is a preferred embodiment of the nanochannel control and sensing circuitry that would support the operation of the nanochannel chip. At left is an array circuit architecture that could control the actuator or sensor array grid that supports a field of nanochannels. In the middle is a schematic of how the nanochannels could be outfitted with dense arrays of motion control electrodes or sensor electrodes for sensing DNA position in the channels electrically. The chip would interface to a Micro Controller Unit (MCU) for high level control of the device, including input and output. It can also have DAC and ADC support for input and output. At lower left and lower right are exemplar circuits, the left is a finite state machine (FSM) for each pixel that can applying one of two voltages for electrode control, and at right is such a circuit that can apply higher voltages (high than the standard core CMOS voltage of ˜3V) to drive the channel electrodes as a mitigation if higher voltages were required.

### Definitions and Interpretations

As used herein, the term “DNA” in various contexts may refer to single stranded or double stranded forms of the molecule, which may refer to the physical material of deoxyribonucleic acid, oligomers of such, or pools of such material, or alternatively to the symbolic sequences for such, in contexts where this makes sense. The term “DNA” may also in various contexts refers not only to strands composed of the four bases A, C, G, T, but also of ribonucleotides such as in RNA, other base analogues, such as U (uracil), I (Inosine), and other well-known universal bases or base analogues or modified or marked bases, including well-known epigenetics marks on bases, such as 5mC (5-methyl-C), as well as dye-labelled bases, or bases modified for future labelling or conjugation, such as biotinylated bases, or thiolate bases, and in general any other widely known modified forms of bases used in DNA oligos, including possible modifications in the sugar or backbone of DNA as well. In addition, where it makes sense in context, the term DNA encompasses other nucleic acid (NA) polymers such as RNA (Ribo-), PNA (Peptide-), LNA (Locked-), and diverse forms of XNA (Xeno-), or other common chemical analogues of DNA or nucleic acid oligomers.

As used herein, the terms “DNA assembly” or “joining DNA” refers to any process for physically connecting together two or more existing DNA molecule strands, with the connection at or near the ends, to produce a single molecule strand. In various contexts, such strands may be entirely single stranded DNA, or entirely double stranded DNA, or DNA that is partially single stranded and double stranded. Such strands that are assemble or joined may be connected by covalent phosphate backbone bonds or may be joined through the hydrogen bonding of complementary regions, or in some contexts may be joined through other chemical reactions and chemical groups, such as carbon chain linkers from the end of one backbone to the start of another.

As used herein, the term “nanochannel” refers to any channel structure that is nanometer (nm) scale in its width and depth dimensions, such as up to 10 nm, or up to 100 nm, or several hundred nm, and substantially longer in its third dimension of length, such as 1000 nm or longer, 10,000 nm or longer, or 100,000 nm or longer, or up to 1 millimeter (mm) or longer, or 10 mm or longer. Such channels may be straight, curved, or branched in various contexts. Such channels may reside in a single plane or may extend into 3D within a material substrate.

As used here in, “Phosphoramidite Synthesis” or “the Phosphoramidite Method” or “Chemical synthesis” refer to any of the family of standard or well-known chemical cycles employed for synthesis using phosphoramidite bases, such as those used for commercial DNA oligo synthesis, or those deriving from the original methods such as put forth by Marvin Carruthers.

As used herein, “DNA synthesis” refers to fabrication of physical DNA through a series of chemical reactions, in accordance with producing a desired specific target sequence or sequences. Depending on the context, and when it is clear by such context, “DNA synthesis” may also refer to the process of DNA assembly by joining together shorter segments to form longer ones.

As used herein, the term “chip” refers to a semiconductor integrated circuit chip. In certain contexts where this is clear, it may refer to a CMOS chip.

As used herein, the term “CMOS”, which is an acronym for “Complementary Metal Oxide Semiconductor”, and refers to chips that are made by the CMOS process.

As used herein, “DNA sequencing” refers to processes for reading the identities of the series of bases in a DNA strand or strands.

As used herein, the term “PCR”, which is an acronym for Polymerase Chain Reaction, generally refers to any means of amplifying or copying DNA, including by thermo-cycling PCR, or isothermal PCR reactions, or generally any other processes that can be used to amplify or copy DNA.

As used herein, the term “Error Correction” means to use various techniques to locate errors in the physical DNA base sequence constructed in the channel (relative to an intended sequence design) and to correct these base errors back to the intended sequence in the physical DNA strand.

1-41. (canceled)

42. A device, comprising:
a nanochannel fabricated in a substrate;
the nanochannel configured to hold a DNA construct in the nanochannel,
wherein the construct is positioned such that a joinable end of the DNA
construct extends outside the nanochannel into an accessible inlet reservoir.

43. The device of claim 42, wherein the joinable end of the DNA construct is provided with a joinable DNA segment that can join to the joinable end.

44. The device of claim 42, wherein the device is configured with control electrodes capable of applying driving forces; wherein the control electrodes are positioned at an end of the nanochannel.

45. The device of claim 44, wherein the control electrodes are embedded along the nanochannel.

46. The device of claim 42, wherein the device is configured with one or more nanoslit fluidic access ports along the nanochannel.

47. The device of claim 43, wherein the DNA construct comprises a shuttle construct having a first blocking group.

48. The device of claim 47, wherein the joinable DNA segment comprises a second blocking group.

49. The device of claim 48, wherein the first and second blocking groups are selected from one or more of: a material bead, and a large biomolecule; and the blocking group comprising a head group, and a body.

50. The device of claim 48, comprising multiple orthogonally cleavable blocking groups.

51. The device of claim 49, wherein the head group comprises one or more of: a material bead, a dye molecule, and a dye structure; and wherein the body comprises one or more of: natural DNA, synthetic DNA, and non-DNA elements.

52. The device of claim 42, comprising replicates of the nanochannel, wherein each replicate shares a common inlet, common outlet reservoirs, and common electrodes.

53. The device of claim 42, wherein the device is provided on a CMOS chip device configured for scalable, parallel, automated long DNA assembly, wherein the nanochannel is fabricated on a surface of the CMOS chip device, and wherein the CMOS chip device comprises circuitry configured to supply control voltages to control electrodes of the nanochannel device.

54. A method for long DNA assembly comprising:
introducing a shuttle construct with a blocking group into the device of claim 42;
contacting a shuttle tail of the shuttle construct with a first joinable DNA segment introduced into the inlet reservoir in a first segment solution;
performing a joining reaction;
flushing out the first segment solution;
cleaving a blocking group from the shuttle construct; and
advancing the shuttle construct within the channel.

55. The method of claim 54, comprising repeating the introducing, contacting, performing, and flushing steps at least one or more times before the cleaving step.

56. The method of claim 54, comprising: repeating the steps of: introducing additional joinable DNA segments, performing the joining reaction, and advancing the shuttle or a growing strand constructed from the joining reaction.

57. The method of claim 54, wherein the joining reaction comprises a hybridization reaction.

58. The method of claim 54, wherein the first joinable DNA segment and the additional joinable DNA segments comprise double stranded DNA in a length range of about 15 bases to about 40 bases, or about 100 bases to about 100 kilobases.

59. The method of claim 54, wherein the first joinable DNA segment and the additional joinable DNA segments comprise a double-stranded portion with a 5′ overhang on a first end, and a 3′ overhang on a second end.

60. The method of claim 54, wherein a final DNA construct is produced, and wherein the final DNA construct is ejected into an agarose gel solution in an output reservoir.

61. The method of claim 54, wherein the method is performed on a set of replicate channels, wherein each channel of the set of replicate channels shares a common inlet, a common outlet, and common control electrodes.


SEQUENCE LISTING (8 sequences)

SEQ ID NO: 1 - DNA, 61 bp, synthetic construct
  source 1..61: mol_type = other DNA
  modified_base 15: mod_base = OTHER; note = Internal deoxyInosine
  modified_base 21: mod_base = OTHER; note = Internal dioxyUridine
gtttttttttttttntttttnttttttcagaatcgttagttgatggcgaaaggtcgcaaag

SEQ ID NO: 2 - DNA, 88 bp, synthetic construct
  source 1..88: mol_type = other DNA
aggtcgccgcccctttgcgacctttcgccatcaactaacgattctgcgttggctacccgtgatattgctgaagagcttggcggcgaat

SEQ ID NO: 3 - DNA, 65 bp, synthetic construct
  source 1..65: mol_type = other DNA
aggtcgccgcccctttgcgacctttcgccatcaactaacgattctgcgttggctacccgtgatat

SEQ ID NO: 4 - DNA, 12 bp, synthetic construct
  source 1..12: mol_type = other DNA
gggcggcgacct

SEQ ID NO: 5 - DNA, 12 bp, synthetic construct
  source 1..12: mol_type = other DNA
aggtcgccgccc

SEQ ID NO: 6 - DNA, 42 bp, synthetic construct
  source 1..42: mol_type = other DNA
  modified_base 11: mod_base = OTHER; note = Internal deoxyInosine
  modified_base 17: mod_base = OTHER; note = Internal dioxyUridine
gtttttttttntttttnttttttcagaatcgttagttgatgg

SEQ ID NO: 7 - DNA, 54 bp, synthetic construct
  source 1..54: mol_type = other DNA
  modified_base 50: mod_base = i
  modified_base 1: mod_base = OTHER; note = Phosphorylated adenine
aggtcgccgcccctttgcgacctttcgccatcaactaacgattctgcgtnggct

SEQ ID NO: 8 - DNA, 57 bp, synthetic construct
  source 1..57: mol_type = other DNA
aggtcgccgcccctttgcgacctttcgccatcaactaacgattctgcgttggctacc